(19) United States
(12) Patent Application Publication
(10) Pub. No.: US 2005/0107437 A1
(43) Pub. Date: May 19, 2005

(54) Novel herbicides

(76) Inventors: Jurgen Schaetzer, Basel (CH); Jean Wenger, Basel (CH); Sabine Berteina-Raboin, Orleans Cedex (FR); Andre Stoller, Basel (CH); Kurt Nebel, Basel (CH); Roger Graham Hall, Basel (CH)

Correspondence Address: SYNGENTA CROP PROTECTION , INC.;PATENT AND TRADEMARK DEPARTMENT, 410 SWING ROAD, GREENSBORO, NC, 27409, US

(21) Appl. No.: 10/497,976

(22) PCT Filed: Dec. 10, 2002
(86) PCT No.: PCT/EP02/14006

(30) Foreign Application Priority Data
Dec. 11, 2001 (CH) 2255/01

(51) Int. Cl. A61K 31/444; C07D 41/02

(52) U.S. Cl. 514/335; 546/261

(57) ABSTRACT

Compounds of formula I wherein the substituents R₁, R₂, R₃, and R₄ and the suffixes n and m are defined in claim 1, and the agrochemically acceptable salts and all stereoisomers and tautomers of those compounds are suitable for use as herbicides.

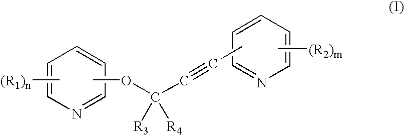

The present invention relates to novel, herbicidally active pyridylalkynes, to processes for their preparation, to compositions comprising those compounds, and to their use in controlling weeds, especially in crops of useful plants, or in inhibiting plant growth.

Phenyl- and pyridyl-alkynes having herbicidal action are described, for example, in JP-A-11 147 866, WO 01/55066, WO 02/28182 and PCT Application No. EP02/08878.

Novel pyridylalkynes having herbicidal and growth-inhibiting properties have now been found.

The present invention accordingly relates to compounds of formula I 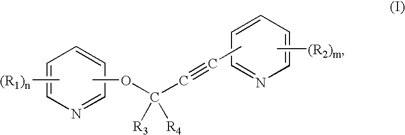
wherein
n is 0, 1, 2, 3 or 4;
each R₁ independently of any others is halogen, —CN, —SCN, —SF₅, —NO₂, —NR₅R₆, —CO₂R₇, —CONR₈R₉, —C(R₁₀)═NOR₁₁, —COR₁₂, —OR₁₃, —SR₁₄, —SOR₁₅, —SO₂R₁₆, —OSO₂R₁₇, C₁-C₉alkyl, C₂-C₈alkenyl, C₂-C₈alkynyl or C₃-C₆cycloalkyl; or is C₁-C₈alkyl, C₂-C₈alkenyl or C₂-C₈alkynyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)=NR₂₈, —OR₂₉, —SR₃₀, —SOR₃₁, —S₂R₃₂ or C₃-C₆cycloalkyl substituents; or
each R₁ independently of any others is C₃-C₆cycloalkyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)=NR₂₈, —SR₃₀, —SOR₃₁, —SO₂R₃₂ or C₃-C₆cycloalkyl substituents; or
each R₁ independently of any others is phenyl, which may in turn be substituted by one or more halogen, C₁-C₄alkyl, C₁-C₄haloalkyl, C₁-C₄alkoxy, —CN, —NO₂, C₁-C₄alkylthio, C₁-C₄alkyl-sulfinyl or C₁-C₄alkylsulfonyl substituents; or
two adjacent R₁ together form a C₁-C₇alkylene bridge, which may be interrupted by 1 or 2 non-adjacent oxygen atoms and may be substituted by C₁-C₆alkyl or C₁-C₆alkoxy, the total number of ring atoms being at least 5 and at most 9; or
two adjacent R₁ together form a C₂-C₇alkenylene bridge, which may be interrupted by 1 or 2 non-adjacent oxygen atoms and may be substituted by C₁-C₆alkyl or C₁-C₆alkoxy, the total number of ring atoms being at least 5 and at most 9;
R₃ and R₄ are each independently of the other hydrogen, halogen, —CN, C₁-C₄alkyl or C₁-C₄-alkoxy; or
R₃ and R₄ together are C₂-C₅alkylene;
R₅ is hydrogen, C₁-C₈alkyl or —C(O)C₁-C₈alkyl;
R₆ is hydrogen, C₁-C₈alkyl, C₃-C₈ alkenyl, C₃-C₈alkynyl, phenyl or benzyl; wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₅ and R₆ together are a C₂-C₅alkylene chain, which may be interrupted by an oxygen or a sulfur atom;
R₇ is hydrogen, C₁-C₈alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, wherein phenyl may in turn be substituted by one or more halogen, C₁-C₄alkyl, C₁-C₄halo-alkyl, C₁-C₄alkoxy, —CN, —NO₂, C₁-C₄alkylthio, C₁-C₄alkylsulfinyl or C₁-C₄alkylsulfonyl substituents;
R₈ is hydrogen or C₁-C₈alkyl;
R₉ is hydrogen or C₁-C₈alkyl, or is C₁-C₈alkyl substituted by one or more —COOH, C₁-C₈alkoxycarbonyl or —CN substituents, or
R₉ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₉ and R₉ together are C₂-C₅alkylene;
R₁₀ is hydrogen, C₁-C₄alkyl, C₁-C₄haloalkyl or C₃-C₆cycloalkyl;
R₁₁ is hydrogen, C₁-C₈alkyl, C₃-C₈alkenyl, C₃-C₈alkynyl, C₁-C₄haloalkyl or C₃-C₆haloalkenyl;
R₁₂ is hydrogen, C₁-C₄alkyl, C₁-C₄haloalkyl or C₃-C₆cycloalkyl;
R₁₃ is hydrogen, C₁-C₈alkyl, C₃-C₈alkenyl or C₃-C₈alkynyl; or
R₁₃ is phenyl or phenyl-C₁-C₆alkyl, wherein the phenyl ring may in turn be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents, or
R₁₃ is C₁-C₈alkyl substituted by one or more halogen, —CN, C₁-C₆alkylamino, di(C₁-C₆alkyl)-amino or C₁-C₄alkoxy substituents;
R₁₄ is hydrogen, C₁-C₈alkyl, C₃-C₈alkenyl or C₃-C₈alkynyl, or is C₁-C₈alkyl substituted by one or more halogen, —CN or C₁-C₄alkoxy substituents;
R₁₅, R₁₆ and R₁₇ are each independently of the others C₁-C₈alkyl, C₃-C₈alkenyl or C₃-C₈-alkynyl, or C₁-C₈alkyl substituted by one or more halogen, —CN or C₁-C₄alkoxy substituents;
R₁₈ is hydrogen or C₁-C₈alkyl;
R₁₉ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₁₈ and R₁₉ together are a C₂-C₅alkylene chain, which may be interrupted by an oxygen or a sulfur atom;
R₂₀ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents;
R₂₁ is hydrogen or C₁-C₈alkyl;
R₂₂ is hydrogen or C₁-C₈alkyl, or is C₁-C₈alkyl substituted by one or more —COOH, C₁-C₈-alkoxycarbonyl or —CN substituents, or
R₂₂ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₂₁ and R₂₂ together are C₂-C₅alkylene;
R₂₃ is hydrogen, C₁-C₄alkyl, C₁-C₄haloalkyl or C₃-C₆cycloalkyl;
R₂₄ is hydrogen, C₁-C₄alkyl, C₁-C₄haloalkyl or C₃-C₆cycloalkyl;
R₂₅ is hydrogen, C₁-C₈alkyl, C₃-C₈alkenyl, C₃-C₈alkynyl, C₁-C₄haloalkyl or C₃-C₆haloalkenyl;
R₂₆ is hydrogen or C₁-C₈alkyl;
R₂₇ is hydrogen or C₁-C₈alkyl, or is C₁-C₈alkyl substituted by one or more —COOH, C₁-C₈-alkoxycarbonyl or —CN substituents, or
R₂₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₂₆ and R₂₇ together are C₂-C₅alkylene;
R₂₈ is hydrogen or C₁-C₈alkyl;
R₂₉ and R₃₀ are each independently of the other hydrogen, C₁-C₈alkyl, C₃-C₈alkenyl or C₃-C₈alkynyl, or C₁-C₈alkyl substituted by one or more halogen, —CN or C₁-C₄alkoxy substituents;
R₃₁ and R₃₂ are each independently of the other C₁-C₈alkyl, C₃-C₈alkenyl or C₃-C₈alkynyl, or C₁-C₈alkyl substituted by one or more halogen, —CN or C₁-C₄alkoxy substituents;
m is 0, 1, 2, 3or 4;
each R₂ independently of any others is halogen, —CN, —SCN, —OCN, —N₃, —SF₅, —NO₂, —NR₃₃R₃₄, —CO₂R₃₅, —CONR₃₆R₃₇, —C(R₃₈)═NOR₃₉, —COR₄₀, —OR₄₁, —SR₄₂, —SOR₄₃, —SO₂R₄₄, —OSO₂R₄₅, —N([CO]ₚR₄₆)COR₄₇, —N(OR₅₄)COR₅₅, —N(R₅₆)SO₂R₅₇, —N(SO₂R₅₈)SO₂R₅₉, —N═C(OR₆₀)R₆₁, —CR₆₂(OR₆₃)OR₆₄, —OC(O)NR₆₅R₆₆, —SC(O)NR₆₇R₆₈, —OC(S)NR₆₉R₇₀ or —N-phthalimide; or
R₂ is a 5- to 7-membered heterocyclic ring system, which may be aromatic or partially or fully saturated and may contain from 1 to 4 hetero atoms selected from nitrogen, oxygen and sulfur, it being possible for that heterocyclic ring system in turn to be substituted by one or more halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or C₁-C₆alkylsulfonyl substituents;
R₃₃ is hydrogen or C₁-C₈alkyl; and
R₃₄ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₃₃ and R₃₄ together are a C₂-C₅alkylene chain, which may be interrupted by an oxygen or a sulfur atom;
R₃₅ is hydrogen, C₁-C₈alkyl, C₃-C₈alkenyl or C₃-C₈alkynyl, or is C₁-C₈alkyl, C₃-C₈ alkenyl or C₃-C₈alkynyl substituted by one or more halogen, C₁-C₄alkoxy or phenyl substituents, wherein phenyl may in turn be substituted by one or more halogen, C₁-C₄alkyl, C₁-C₄halo-alkyl, C₁-C₄alkoxy, —CN, —NO₂, C₁-C₄alkylthio, C₁-C₄alkylsulfinyl or C₁-C₄alkylsulfonyl substituents;
R₃₆ is hydrogen or C₁-C₈alkyl;
R₃₇ is hydrogen or C₁-C₈alkyl, or is C₁-C₈alkyl substituted by one or more —COOH, C₁-C₈-alkoxycarbonyl or —CN substituents, or
R₃₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₃₆ and R₃₇ together are C₃-C₅alkylene;
R₃₈ is hydrogen, C₁-C₄alkyl, C₁-C₄haloalkyl or C₃-C₆cycloalkyl;
R₃₉ is hydrogen, C₁-C₈alkyl, C₃-C₈alkenyl, C₃-C₈alkynyl, C₁-C₄haloalkyl or C₃-C₆haloalkenyl;
R₄₀ is hydrogen, C₁-C₄alkyl, C₁-C₄haloalkyl, C₁-C₈alkylthio, —C(O)—C(O)OC₁-C₄alkyl or C₃-C₆-cycloalkyl;
R₄₁ is hydrogen, C₁-C₈alkyl, C₃-C₈alkenyl, C₃-C₈alkynyl, C₁-C₆alkoxy-C₁-C₆alkyl, C₁-C₈alkyl-carbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or
R₄₁ is phenyl or phenyl-C₁-C₆alkyl, wherein the phenyl ring may in turn be substituted by one or more halogen, C₁-C₄alkyl, C₁-C₄haloalkyl, C₁-C₄alkoxy, —CN, —NO₂ or —S(O)₂C₁-C₈alkyl substituents, or
R₄₁ is C₁-C₈alkyl substituted by one or more halogen, —COOH, C₁-C₈alkoxycarbonyl, C₁-C₆alkylamino, di(C₁-C₆alkyl)amino or —CN substituents;
R₄₂ is hydrogen, C₁-C₈alkyl, C₃-C₈alkenyl or C₃-C₈alkynyl, or is C₁-C₈alkyl substituted by one or more halogen, —CN or C₁-C₄alkoxy substituents;
R₄₃ and R₄₄ are each independently of the other C₁-C₈alkyl, C₃-C₈alkenyl or C₃-C₈alkynyl, or C₁-C₈alkyl substituted by one or more halogen, —CN or C₁-C₄alkoxy substituents;
R₄₅ is C₁-C₈alkyl, C₁-C₈alkyl substituted by one or more halogen, —CN or C₁-C₄alkoxy substituents, C₃-C₈alkenyl or C₃-C₈alkynyl, or
R₄₅ is phenyl, it being possible for the phenyl ring to be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;
R₄₅ is hydrogen, C₁-C₈alkyl, C₃-C₈alkenyl, C₃-C₈alkynyl or C₁-C₄haloalkyl;
R₄₇ is hydrogen, C₁-C₈alkyl, C₁-C₄alkoxy, C₃-C₈alkenyl or C₃-C₈alkynyl, or is C₁-C₈alkyl substituted by one or more halogen, —CN, C₁-C₄alkoxy, C₁-C₈alkoxycarbonyl, —NH₂, C₁-C₄-alkylamino, di(C₁-C₄alkyl)amino, —NR₄₈COR₄₉, —NR₅₀SO₂R₅₁ or —NR₅₂CO₂R₅₃ substituents, or
R₄₇ is phenyl or benzyl, each of which may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
p is 0 or 1;
R₄₈, R₄₉, R₅₀, R₅₁, R₅₂ and R₅₃ are each independently of the others hydrogen, C₁-C₈alkyl, phenyl, benzyl or naphthyl, it being possible for the three last-mentioned aromatic radicals in turn to be substituted by one or more halogen, C₁-C₈alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;
R₅₄ and R₅₅ are each independently of the other hydrogen, C₁-C₈alkyl or phenyl, which may in turn be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;
R₅₆ is hydrogen, C₁-C₈alkyl, C₁-C₄haloalkyl, C₁-C₄alkoxy, C₃-C₈alkenyl, C₃-C₈alkynyl or benzyl, it being possible for benzyl in turn to be substituted by one or more halogen,
C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;
R₅₇ is C₁-C₈alkyl, C₁-C₄haloalkyl, phenyl, benzyl or naphthyl, it being possible for the three last-mentioned aromatic rings to be substituted by one or more halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;
R₆₈ and R₅₉ are each independently of the other C₁-C₈alkyl, C₃-C₈alkenyl, C₃-C₈alkynyl, phenyl, benzyl or naphthyl, it being possible for the three last-mentioned aromatic rings to be substituted by one or more halogen, C₁-C₄alkyl, C₁-C₄haloalkyl, C₁-C₄alkoxy, C₁-C₄alkyl-amino, di(C₁-C₄alkyl)amino, —NH₂, —CN, —NO₂, C₁-C₄alkylthio, C₁-C₄alkylsulfinyl or C₁-C₄alkyl-sulfonyl substituents;
R₆₀ and R₆₁ are each independently of the other hydrogen or C₁-C₆alkyl;
R₆₂, R₆₃ and R₆₄ are each independently of the others hydrogen or C₁-C₈alkyl, or R₆₃ and R64 together form a C₂-C₅alkylene bridge;
R₆₅, R₆₆, R₆₇, R₆₈, R₆₉ and R₇₀ are each independently of the others hydrogen or C₁-C₈alkyl, or
R₈₆ and R₆₆ together or R₆₇ and R₆₈ together or R₆₉ and R₇₀ together form a C₂-C₅alkylene bridge; or
each R₂ independently of any others is C₁-C₈alkyl, or is C₁-C₈alkyl mono- or poly-substituted by halogen, —CN, —N₃, —SCN, —NO₂, —NR₇₁R₇₂, —CO₂R₇₃, —CONR₇₄R₇₅, —COR₇₆, —C(R₇₇)═NOR₇₈, —C(S)NR₇₉R₈₀, —C(C₁-C₄alkylthio)=NR₈₁, —OR₈₂, —SR₈₃, —SOR₈₄, —SO₂R₈₅, —O(SO₂)R₈₆, —N(R₈₇)CO₂R₈₈, —N(R₈₉)COR₉₀, —S⁺(R₉₁)₂, —N⁺(R₉₂)₃, —Si(R₉₃)₃ or C₃-C₆cycloalkyl; or
each R₂ independently of any others is C₁-C₈alkyl substituted by a 5- to 7-membered heterocyclic ring system, which may be aromatic or partially or fully saturated and may contain from 1 to 4 hetero atoms selected from nitrogen, oxygen and sulfur, it being possible for that heterocyclic ring system in turn to be substituted by one or more halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or C₁-C₆alkylsulfonyl substituents; or
each R₂ independently of any others is C₂-C₈alkenyl, or is C₂-C₈alkenyl mono- or poly-substituted by —CN, —NO₂, —CO₂R₉₄, —CONR₉₅R₉₆, —COR₉₇, —C(R₉₈)═NOR₉₉, —C(S)NR₁₀₀R₁₀₁, —C(C₁-C₄alkylthio)=NR₁₀₂, —OR₁₀₃, —Si(R₁₀₄)₃ or C₃-C₆cycloalkyl; or
each R₂ independently of any others is C₂-C₈alkynyl, or is C₂-C₈alkynyl mono- or poly-substituted by halogen, —CN, —CO₂R₁₀₅, —CONR₁₀₆R₁₀₇, —COR₁₀₈, —C(R₁₀₉)═NOR₁₁₀, —C(S)NR₁₁₁R₁₁₂, —C(C₁-C₄alkylthio)=NR,₁₁₃, —OR₁₁₄, —Si(R₁₁₅)₃ or C₃-C₆cycloalkyl; or each R₂ independently of any others is C₃-C₆cycloalkyl, or is C₃-C₈cycloalkyl mono- or poly-substituted by halogen, —CN, —CO₂R₁₁₆, —CONR₁₁₇R₁₁₈, —COR₁₁₉, —C(R₁₂₀)═NOR₁₂₁, —C(S)NR₁₂₂R₁₂₃ or —C(C₁-C₄alkylthio)=NR₁₂₄; or
two adjacent R₂ together form a C₁-C₇alkylene bridge, which may be interrupted by 1 or 2 non-adjacent oxygen atoms and may be substituted by cyano, C₁-C₆alkyl or C₁-C₆alkoxy, the total number of ring atoms being at least 5 and at most 9; or
two adjacent R₂ together form a C₂-C₇alkenylene bridge, which may be interrupted by 1 or 2 non-adjacent oxygen atoms and may be substituted by cyano, C₁-C₆alkyl or C₁-C₆alkoxy, the total number of ring atoms being at least 5 and at most 9;
R₇₁ is hydrogen or C₁-C₈alkyl;
R₇₂ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₇₁ and R₇₂ together are a C₂-C₅alkylene chain, which may be interrupted by an oxygen or a sulfur atom;
R₇₃ is hydrogen, C₁-C₁alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, it being possible for phenyl in turn to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkyl-sulfonyl substituents;
R₇₄ is hydrogen or C₁-C₈alkyl;
R₇₅ is hydrogen, C₁-C₈alkyl or C₃-C₇cycloalkyl, or is C₁-C₈alkyl substituted by one or more —COOH, C₁-C₈alkoxycarbonyl, C₁-C₆alkoxy or —CN substituents; or
R₇₅ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₇₄ and R₇₅ together are a C₂-C₅alkylene chain, which may be interrupted by an oxygen or sulfur atom;
R₇₆ is hydrogen, C₁-C₄alkyl, C₁-C₄haloalkyl or C₃-C₆cycloalkyl;
R₇₇ is hydrogen, C₁-C₄alkyl, C₁-C₄haloalkyl or C₃-C₆cycloalkyl;
R₇₈ is hydrogen, C₁-C₈alkyl, C₃-C₈alkenyl, C₃-C₈alkynyl, C₁-C₄haloalkyl or C₃-C₆haloalkenyl; and
R₇₉ is hydrogen or C₁-C₈alkyl;
R₈₀ is hydrogen or C₁-C₈alkyl, or is C₁-C₈alkyl substituted by one or more —COOH, C₁-C₈-alkoxycarbonyl or —CN substituents; or
R₈₀ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₇₉ and R₈₀ together are C₂-C₅alkylene;
R₈₁ is hydrogen or C₁-C ₈alkyl;
R₈₂ is —Si(C₁-C₆alkyl)₃, C₃-C₈alkenyl, C₃-C₈alkynyl or C₁-C₈alkyl, which is mono- or poly-substituted by halogen, —CN, —NH₂, C₁-C₆alkylamino, di(C₁-C₆alkyl)amino or C₁-C₄alkoxy;
R₈₃ is hydrogen, C₁-C₈alkyl, C₃-C₈alkenyl, C₃-C₈alkynyl or C₁-C₈alkyl, which is mono- or poly-substituted by halogen, —CN, —NH₂, C₁-C₆alkylamino, di(C₁-C₆alkyl)amino or C₁-C₄alkoxy;
R₈₄, R₈₅ and R₈₆ are each independently of the others C₁-C₈alkyl, C₃-C₈alkenyl or C₃-C₈-alkynyl, or C₁-C₈alkyl which is substituted by one or more halogen, —CN or C₁-C₄alkoxy substituents;
R₈₇ and R₈₉ are each independently of the other hydrogen, C₁-C₈alkyl or C₁-C₈alkoxy;
R₈₈ is C₁-C₈alkyl;
R₉₀ is hydrogen or C₁-C₈alkyl;
R₉₁ is C₁-C₄alkyl;
R₉₂ and R₉₃ are each independently of the other C₁-C₆alkyl;
R₉₄ is hydrogen, C₁-C₈alkyl, C₃-C₈alkenyl or C₃-C₈alkynyl, wherein the last 3 substituents may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₉₅ is hydrogen or C₁-C₈alkyl;
R₉₆ is hydrogen or C₁-C₈alkyl, or is C₁-C₈alkyl substituted by one or more —COOH, C₁-C₈-alkoxycarbonyl or —CN substituents; or
R₉₆ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₉₅ and R₉₆ together are C₂-C₅alkylene;
R₉₇ and R₉₈ are each independently of the other hydrogen, C₁-C₄alkyl, C₁-C₄haloalkyl or C₃-C₆cycloalkyl;
R₉₉ is hydrogen, C₁-C₈alkyl, C₃-C₈alkenyl, C₃-C₈alkynyl, C₁-C₄haloalkyl or C₃-C₆haloalkenyl;
R₁₀₀ is hydrogen or C₁-C₈alkyl;
R₁₀₁ is hydrogen or C₁-C₈alkyl, or is C₁-C₈alkyl substituted by one or more —COOH, C₁ -C₈-alkoxycarbonyl or —CN substituents; or
R₁₀₁ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₀₀ and R₁₀₁ together are C₂-C₅alkylene;
R₁₀₂ is hydrogen or C₁-C₈alkyl;
R₁₀₃ is hydrogen, C₁-C₈ alkyl, —Si(C₁-C₆alkyl)₃, C₃-C₈alkenyl or C₃-C₈alkynyl;
R₁₀₄ is C₁-C₆alkyl;
R₁₀₅ is hydrogen, C₁-C₈alkyl, C₃-C₈alkenyl or C₃-C₈alkynyl, wherein the last 3 substituents may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₁₀₆ is hydrogen or C₁-C₈alkyl;
R₁₀₇ is hydrogen or C₁-C₈alkyl, or is C₁-C₈alkyl substituted by one or more —COOH, C₁-C₈-alkoxycarbonyl or —CN substituents; or
R₁₀₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₀₆ and R₁₀₇ together are C₂-C₅alkylene;
R₁₀₈ is hydrogen, C₁-C₄alkyl, C₁-C₄haloalkyl or C₃-C₆cycloalkyl;
R₁₀₉ is hydrogen, C₁-C₄alkyl, C₁-C₄haloalkyl or C₃-C₆cycloalkyl;
R₁₁₀ is hydrogen, C₁-C₈alkyl, C₃-C₈alkenyl, C₃-C₈alkynyl, C₁-C₄haloalkyl or C₃-C₆haloalkenyl;
R₁₁₁, is hydrogen or C₁-C₈alkyl;
R₁₁₂ is hydrogen or C₁-C₈alkyl, or is C₁-C₈alkyl substituted by one or more —COOH, C₁-C₈-alkoxycarbonyl or —CN substituents; or
R₁₁₂ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₁₁ and R₁₁₂ together are C₂-C₅alkylene;
R₁₁₃ is hydrogen or C₁-C₈alkyl;
R₁₁₄ is hydrogen, C₁-C₈alkyl, —Si(C₁-C₆alkyl)₃, C₃-C₈alkenyl or C₃-C₈alkynyl;
R₁₁₅ is C₁-C₆alkyl;
R₁₁₆ is hydrogen, C₁-C₈alkyl, C₃-C₈alkenyl or C₃-C₈alkynyl, wherein the last 3 substituents may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₁₁₇ is hydrogen or C₁-C₈alkyl;
R₁₁₈ is hydrogen or C₁-C₈alkyl, or is C₁-C₈alkyl substituted by one or more —COOH, C₁-C₈-alkoxycarbonyl or —CN substituents; or
R₁₁₈ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₁₇ and R₁₁₈ together are C₂-C₅alkylene;
R₁₁₉ is hydrogen, C₁-C₄alkyl, C₁-C₄haloalkyl or C₃-C₆cycloalkyl; and
R₁₂₀ is hydrogen, C₁-C₄alkyl, C₁-C₄haloalkyl or C₃-C₆cycloalkyl;
R₁₂₁ is hydrogen, C₁-C₈alkyl, C₃-C₈alkenyl, C₃-C₈alkynyl, C₁-C₄haloalkyl or C₃-C₆haloalkenyl;
R₁₂₂ is hydrogen or C₁-C₈alkyl;
R₁₂₃ is hydrogen or C₁-C₈alkyl, or is C₁-C₈alkyl substituted by one or more —COOH, C₁-C₈-alkoxycarbonyl or —CN substituents; or
R₁₂₃ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R122 and R₁₂₃ together are C₂-C₅alkylene; and
R₁₂₄ is hydrogen or C₁-C₈alkyl,
and to the agrochemically acceptable salts and all stereoisomers and tautomers of the compounds of formula I.

When n is 0, all the free valencies on the pyridyl ring of the compounds of formula I are substituted by hydrogen. When m is 0, all the free valencies on the pyridyl ring of the compounds of formula I are substituted by hydrogen.

Examples of substituents that are formed when R₅ and R₆ together or R₁₈ and R₁₉ together or R₃₃ and R₃₄ together or R₇₁, and R₇₂ together or R₇₄ and R₇₅ together are a C₂-C₅alkylene chain, which may be interrupted by an oxygen or a sulfur atom, are piperidine, morpholine, thiomorpholine and pyrrolidine.

Examples of heterocyclic ring systems, which may be aromatic or partially or fully saturated, in the definition of R₂ are:

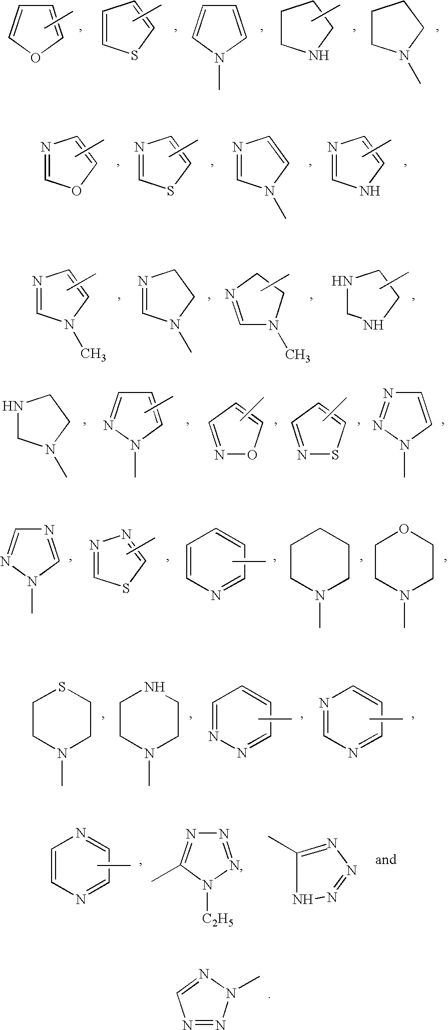

The alkyl groups appearing in the definitions of substituents may be straight-chain or branched and are, for example, methyl, ethyl, n-propyl, isopropyl, n-butyl, sec-butyl, isobutyl, tert-butyl, and also the pentyl, hexyl, heptyl and octyl isomers.

Halogen is fluorine, chlorine, bromine and iodine, preferably fluorine and chlorine. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably trichloro-methyl, difluorochloromethyl, difluoromethyl, trifluoromethyl and dichlorofluoromethyl.

Alkoxy groups have preferably a chain length of from 1 to 6, especially from 1 to 4, carbon atoms. Alkoxy is, for example, methoxy, ethoxy, propoxy, isopropoxy, n-butoxy, isobutoxy, sec-butoxy and tert-butoxy, and also the pentyloxy and hexyloxy isomers; preferably methoxy and ethoxy.

Alkoxy, alkenyl, alkynyl, alkoxyalkyl, alkylthio, alkylsulfonyl, alkylsulfinyl, alkylaminoalkoxy, alkoxycarbonyl, alkylcarbonyloxy, alkenylthio, alkenylsulfonyl, alkenylsulfinyl, alkynylsulfonyl, alkynylthio and alkynylsulfinyl groups are derived from the mentioned alkyl radicals. The alkenyl and alkynyl groups can be mono- or poly-unsaturated. Alkenyl is to be understood as being, for example, vinyl, allyl, methallyl, 1-methylvinyl or but-2-en-1-yl. Alkynyl is, for example, ethynyl, propargyl, but-2-yn-1-yl, 2-methylbutyn-2-yl or but-3-yn-2-yl.

Alkylthio groups have preferably a chain length of from 1 to 4 carbon atoms. Alkylthio is, for example, methylthio, ethylthio, propylthio, isopropylthio, n-butylthio, isobutylthio, sec-butylthio or tert-butylthio, preferably methylthio and ethylthio. Alkylsulfinyl is, for example, methyl-sulfinyl, ethylsulfinyl, propylsulfinyl, isopropylsulfinyl, n-butylsulfinyl, isobutylsulfinyl, sec-butylsulfinyl or tert-butylsulfinyl; preferably methylsulfinyl or ethylsulfinyl. Alkylsulfonyl is, for example, methylsulfonyl, ethylsulfonyl, propylsulfonyl, isopropylsulfonyl, n-butylsulfonyl, isobutylsulfonyl, sec-butylsulfonyl or tert-butylsulfonyl; preferably methylsulfonyl or ethyl-sulfonyl.

Alkoxyalkyl groups have preferably from 1 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

The cycloalkyl groups preferably have from 3 to 6 ring carbon atoms, such as, for example, cyclopropyl, cyclobutyl, cyclopentyl and cyclohexyl. These cycloalkyl groups may be poly-substituted, especially mono- to tri-substituted, by the substituents mentioned, such as, for example, halogen.

As haloalkenyl, mono- or poly-halo-substituted alkenyl groups are suitable, the halogen being fluorine, chlorine, bromine or iodine, especially fluorine or chlorine, for example 2,2-difluoro-1-methylvinyl, 3-fluoropropenyl, 3-chloropropenyl, 3-bromopropenyl, 2,3,3-trifluoropropenyl, 2,3,3-trichloropropenyl and 4,4,4-trifluorobut-2-en-1-yl. Among the mono-, di- or tri-halo-substituted C₃-C₆alkenyl groups preference is given to those having a chain length of from 3 to 5 carbon atoms.

Alkylcarbonyl is preferably acetyl or propionyl.

Alkoxycarbonyl is, for example, methoxycarbonyl, ethoxycarbonyl, propoxycarbonyl, isopropoxycarbonyl, n-butoxycarbonyl, isobutoxycarbonyl, sec-butoxycarbonyl or tert-butoxycarbonyl; preferably methoxycarbonyl or ethoxycarbonyl.

Alkenyloxycarbonyl is, for example, allyloxycarbonyl, methallyloxycarbonyl, but-2-en-1-yl-oxycarbonyl, pentenyloxycarbonyl and 2-hexenyloxycarbonyl.

Hydroxyalkyl is, for example, hydroxymethyl, 2-hydroxyethyl or 3-hydroxypropyl.

Alkylamino is, for example, methylamino, ethylamino, n-propylamino, isopropylamino or the butylamino isomers.

Dialkylamino is, for example, dimethylamino, methylethylamino, diethylamino, n-propyl-methylamino, dibutylamino and diisopropylamino. Preference is given to alkylamino groups having a chain length of from 1 to 4 carbon atoms.

Phenyl, including phenyl as part of a substituent such as benzyl or phenylalkyl, may be in substituted form, in which case the substituents may be in the ortho-, meta- and/or para-position(s). Preferred substituent positions are the ortho- and para-positions to the ring attachment position.

Corresponding meanings may also be given to the substituents in combined definitions, for example alkoxy-alkoxycarbonyl, alkylthio-alkyl, alkylsulfinyl-alkyl, alkylsulfonyl-alkyl and alkoxy-alkyl.

Substituents wherein two adjacent R₁ together form a C₁-C₇alkylene bridge, which may be interrupted by 1 or 2 non-adjacent oxygen atoms and may be substituted by C₁-C₆alkyl or C₁-C₆alkoxy, the total number of ring atoms being at least 5 and at most 9, or two adjacent R₁ together form a C₂-C₇alkenylene bridge, which may be interrupted by 1 or 2 non-adjacent oxygen atoms and may be substituted by C₁-C₆alkyl or C₁-C₆alkoxy, the total number of ring atoms being at least 5 and at most 9, have, for example, the following structures:

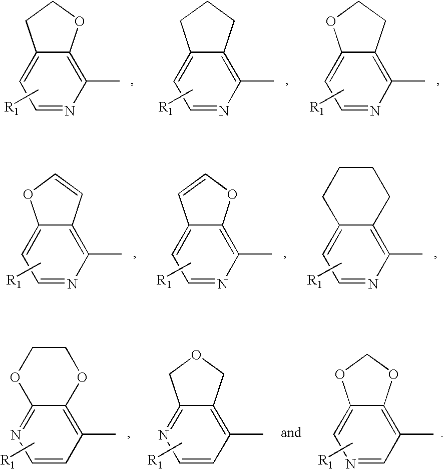

Substituents wherein two adjacent R₂ together form a C₁-C₇alkylene bridge, which may be interrupted by 1 or 2 non-adjacent oxygen atoms and may be substituted by cyano, C₁-C₆-alkyl or C₁-C₆alkoxy, the total number of ring atoms being at least 5 and at most 9, or two adjacent R₂ together form a C₂-C₇alkenylene bridge, which may be interrupted by 1 or 2 non-adjacent oxygen atoms and may be substituted by cyano, C₁-C₆alkyl or C₁-C₆alkoxy, the total number of ring atoms being at least 5 and at most 9, have, for example, the following structures:

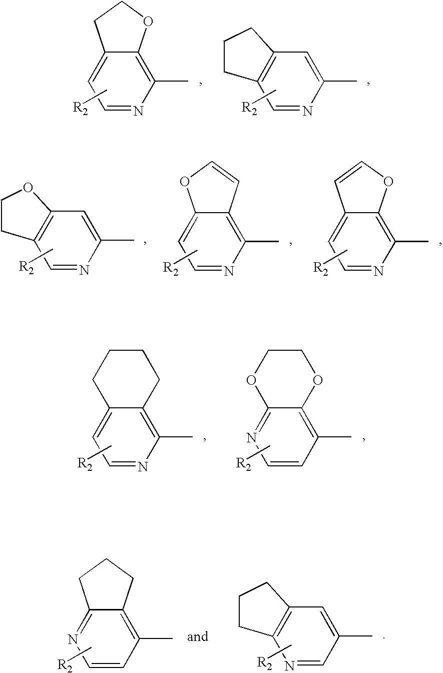

In the definitions of R₁, for example, the phrases “. . . cycloalkyl substituted by one or more halogen, —CN, —NO₂, . . . substituents” and “independently of any others is phenyl, which may in turn be substituted by one or more halogen, C₁-C₄alkyl, C₁-C₄haloalkyl . . . substituents” are to be understood as meaning that the cycloalkyl and phenyl, respectively, can be mono- or poly-substituted, up to and including per-substituted, especially mono- to tri-substituted, by the mentioned substituents, wherein, for halogen, per-halogenation such as, for example, in the case of pentafluorophenyl may also be a preferred pattern of substitution.

This is also true analogously for the definitions of R₆, R₇, R₉, R₁₃, R₁₅, R₁₆, R₁₇, etc., such as, for example: “. . . is C₁-C₈alkyl, C₃-C₈alkenyl or C₃-C₈alkynyl substituted by one or more halogen, C₁-C₄alkoxy or phenyl substituents.

In the definition of R₂, for example, the phrase: “R₂ is a 5- to 7-membered heterocyclic ring system . . . , it being possible for that heterocyclic ring system in turn to be substituted by one or more halogen, C₁-C₄alkyl, C₁-C₄haloalkyl, . . . substituents” means that such heterocyclic ring systems may be especially mono- to tri-substituted at the ring carbon atoms by the mentioned substituents.

The invention relates also to the salts which the compounds of formula I are able to form especially with amines, alkali metal and alkaline earth metal bases or quaternary ammonium bases. Suitable salt-formers are described, for example, in WO 98/41089.

The invention relates also to the salts which the compounds of formula I are able to form with amines, alkali metal and alkaline earth metal bases or quaternary ammonium bases. Among the alkali metal and alkaline earth metal hydroxides as salt formers, special mention should be made of the hydroxides of lithium, sodium, potassium, magnesium and calcium, but especially the hydroxides of sodium and potassium.

Examples of amines suitable for ammonium salt formation include ammonia as well as primary, secondary and tertiary C₁-C₁₈alkylamines, C₁-C₄hydroxyalkylamines and C₂-C₄-alkoxyalkylamines, for example methylamine, ethylamine, n-propylamine, isopropylamine, the four butylamine isomers, n-amylamine, isoamylamine, hexylamine, heptylamine, octyl-amine, nonylamine, decylamine, pentadecylamine, hexadecylamine, heptadecylamine, octa-decylamine, methylethylamine, methylisopropylamine, methylhexylamine, methylnonylamine, methylpentadecylamine, methyloctadecylamine, ethylbutylamine, ethylheptylamine, ethyloctylamine, hexylheptylamine, hexyloctylamine, dimethylamine, diethylamine, di-n-propylamine, diisopropylamine, di-n-butylamine, di-n-amylamine, diisoamylamine, dihexylamine, diheptylamine, dioctylamine, ethanolamine, n-propanolamine, isopropanol-amine, N,N-diethanolamine, N-ethylpropanolamine, N-butylethanolamine, allylamine, n-butenyl-2-amine, n-pentenyl-2-amine, 2,3-dimethylbutenyl-2-amine, dibutenyl-2-amine, n-hexenyl-2-amine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylenediamines, benzidines, naphthylamines and o-, m- and p-chloroanilines; but especially triethylamine, isopropylamine and diisopropylamine.

Preferred quaternary ammonium bases suitable for salt formation correspond e.g. to the formula [N(Rₐ R_{b}R_{c}R_{d})]OH wherein Rₐ, R_{b}, R_{c} and R_{d} are each independently of the others C₁-C₄alkyl. Other suitable tetraalkylammonium bases with other anions can be obtained, for example, by anion exchange reactions.

Preference is given to compounds of formula I wherein R₄₁ is hydrogen, C₁-C₈alkyl, C₃-C₈-alkenyl, C₃-C₈alkynyl, C₁-C₆alkoxy-C₁-C₆alkyl, C₁-C₈alkylcarbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or
R₄₁ is phenyl or phenyl-C₁-C₆alkyl, wherein the phenyl ring may in turn be substituted by one or more halogen, C₁-C₄alkyl, C₁-C₄haloalkyl, C₁-C₄alkoxy, —CN, —NO₂ or —S(O)₂C₁-C₈alkyl substituents, or
R₄₁ is C₁-C₈alkyl substituted by one or more —COOH, C₁-C₈alkoxycarbonyl, C₁-C₆alkylamino, di(C₁-C₆alkyl)amino or —CN substituents.

Preference is also given to compounds of formula I wherein each R₁ independently of any others is halogen, —CN, —NO₂, —C(R₁₀)═NOR₁₁, —OR₁₃, —S(O)₂R₁₆, C₁-C₈alkyl or C₂-C₈alkenyl; or C₁-C₈alkyl substituted by one or more halogen or —CN substituents; n is 0, 1, 2, 3 or 4; and R₁₀, R₁₁, R₁₃ and R₁₆ are as defined for formula 1. Of those compounds, special preference is given to those wherein n is 1, 2 or 3.

Of particular interest are compounds of formula I wherein the group 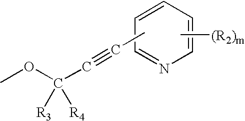
occupies the 2-position on the pyridine ring.

Special preference is given to compounds of formula I wherein each R₁ independently of any others is halogen, —CN, —NO₂, —C(R₁₀)═NOR₁₁, —OR₁₃, —SO₂R₁₆, C₁-C₈alkyl or C₂-C₈alkenyl; or C₁-C₈alkyl substituted by one or more halogen or —CN substituents; n is 1 or 2; R₁₀, R₁₁, R₁₃ and R₁₆ are as defined in claim 1; and the group 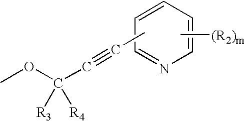
the 2-position on the pyridine ring. Of those, very special preference is given to those compounds wherein R₁ is —CN, —C(R₁₀)═NOR₁, —OR₁₃, C₁-C₄alkyl; or C₁-C₄alkyl substituted by one or more halogen or —CN substituents, and R₁₀ is hydrogen or C₁-C₄alkyl, R₁₁ is hydrogen, C₁-C₄alkyl or C₁-C₄haloalkyl, and R₁₃ is C₁-C₄alkyl, C₃- or C₄-alkenyl, C₃- or C₄-alkynyl, or C₁-C₄alkyl substituted by one or more halogen substituents.

Preference is likewise given to compounds of formula I wherein each R₂ independently of any others is —CN, —SCN, —OCN, —N₃, —CONR₃₆R₃₇, —C(R₃₈)═NOR₃₉, —COR₄₀, —OR₄₁, —OSO₂R₄₅, —N([CO]ₚR₄₆)COR₄₇, —N(R₅₆)SO₂R₅₇, —N(SO₂R₅₈)SO₂R₅₉, —N═C(OR₆₀)R₆₁, or C₁-C₈alkyl; or C₁-C₈alkyl mono- or poly-substituted by halogen, —CN, —N₃, —SCN, —CONR₇₄R₇₅, —COR₇₆, —C(R₇₇)═NOR₇₈, —C(S)NR₇₉R₈₀, —OR₈₂, —SOR₈₄, —SO₂R₈₅ or by —N(R₈₉)COR₉₀; m is 0, 1, 2, 3 or 4; and R₃₆ to R₄₁, R₄₅ to R₄₇, R₅₆ to R₆₁, R₇₄ to R₈₀, R₈₂, R₈₄, R₈₅, R₈₉, R₉₀ and p are as defined for formula 1. Of those compounds, special preference is given to those wherein m is 1 or 2.

Especially preferred compounds of formula I are those wherein each R₂ independently of any others is —CN, —SCN, —OCN, —N₃, —CONR₃₆R₃₇, —OR₄₁, —C(R₃₈)═NOR₃₉ or C₁-C₈alkyl; or C₁-C₈alkyl mono- or poly-substituted by halogen, —CN, —N₃, —SCN or by —C(S)NR₇₉R₈₀; m is 0, 1, 2, 3 or 4, and R₃₆ is hydrogen or C₁-C₄alkyl; R₃₇ is C₁-C₄alkyl or C₁-C₄alkyl one or more C₁-C₄alkoxycarbonyl or —CN substituents; R₃₈ is hydrogen or C₁-C₄alkyl; R₃₉ is hydrogen, C₁-C₄alkyl or C₁-C₄haloalkyl; and R₄₁ is C₁-C₄alkyl, C₃- or C₄-alkenyl, C₃- or C₄-alkynyl, phenyl; or phenyl substituted by one or more halogen or —CN substituents. Of those compounds, very special preference is given to those wherein R₂ is —SCN, —OCN, —N₃, C₁-C₄alkyl; or C₁-C₄alkyl mono- or poly-substituted by halogen, —CN, —N₃ or by —SCN.

The compounds of formula I can be prepared by methods known per se described, for example, in J. Org. Chem. 62,1491-1500 (1997); idem 66, 605-608 (2001); idem 62, 2774-2781 (1997); idem 63,1109-1118 (1998); Tetrahedron Organic Chemistry 2000 (20), 209-213; Synlett 2001(5), 649-651; and K. Sonogashira, Comprehensive Organic Synthesis 1991, Vol. 3, page 521, for example by reacting a compound of formula II 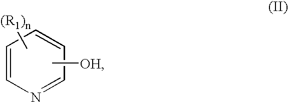
wherein R₁ and n are as defined for formula I, in the presence of a base, with a compound of formula III 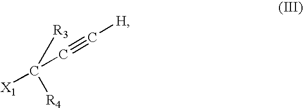
wherein R₃ and R₄ are as defined for formula I and X₁ is O-tosyl, O-mesyl, chlorine, bromine or iodine, to form a compound of formula IV 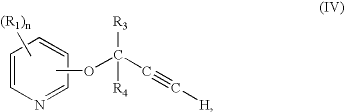
wherein R₁, R₃, R₄ and n are as defined for formula I, and then coupling that compound with a compound of formula V 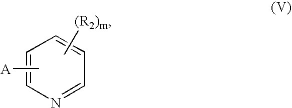
wherein R₂ and m are as defined for formula I and A is a leaving group, e.g. halogen or trifluoromethanesulfonate, in the presence of a palladium catalyst.

The preparation of the compounds of formula I can be carried out, for example, according to the individual Schemes 1, 2, 3, 4 and 5. For the individual synthesis schemes it is generally true that various substituents R₁ and/or R₂ are either already present in compounds of formulae II and/or V at the outset or can be introduced later, for example by nucleophilic or electrophilic aromatic substitution.

According to Reaction Scheme 1, the compounds of formula I can be obtained, for example, from substituted pyridyl propargyl ethers of formula IV.

The pyridyl propargyl ethers of formula IV can be obtained beforehand by etherification of hydroxypyridines of formula II, which are reacted in the presence of a base with acetylene derivatives of formula III. Such etherification reactions are standard procedures and can be carried out, for example, analogously to J. Chem. Soc., Perkin Trans 1, 1979, 2756-2761; Synth. Communic. 18,1111-1118 (1998); J. Chem. Soc., Chem. Communic. 1990, 297-300; J. Org. Chem. 61, 4258-4261 (1996); and Synth. Communic.24, 1367-1379 (1994).

In the next step, the propargyl ethers of formula IV are coupled with substituted pyridine derivatives of formula V under typical Sonogashira conditions (K. Sonogashira, Comprehensive Organic Synthesis 1991, Vol. 3, page 521; J. Org. Chem. 1998 (63), 8551 -8553). Catalyst mixtures that come into consideration are, for example, tetrakistriphenyl-phosphine-palladium or bistriphenylphosphine-palladium dichloride together with copper Iodide, and bases that come into consideration (for the reductive elimination) are especially amines, for example triethylamine, diethylamine and diisopropylethylamine.

The pyridines of formula V preferably carry a leaving group A, wherein A is, for example, halogen or trifluoromethanesulfonate (Tetrahedron Organic Chemistry 2000 (20), 209-213; J. Org. Chem. 63,1109-1118 (1998); Tetrahedron Lett. 27(10), 1171-1174 (1986)). As solvents for the Sonogashira reaction there are customarily used ethers, for example tetrahydrofuran or dioxane, chlorinated hydrocarbons, for example chloroform, or dipolar aprotic solvents, for example dimethylformamide or dimethyl sulfoxide, or amines, for example triethylamine or piperidine.

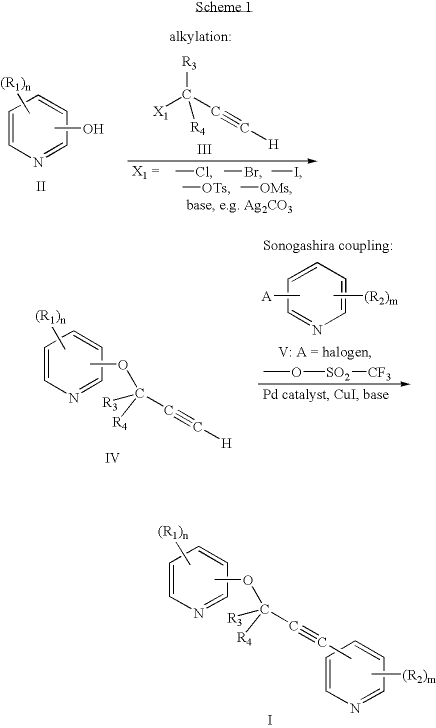

The Pd-catalysed cross-coupling of suitably substituted pyridine derivatives of formula V with terminal acetylenes, for example with propargyl alcohols of formula VI, as shown diagrammatically in Reaction Scheme 2, is known generally as the Sonogashira reaction. That reaction is documented, for example, in Tetrahedron Organic Chemistry 2000 (20), 209-213; Synthesis 1984, 571; and J. Org. Chem. 53, 386 (1988) and can likewise be used for the preparation of the pyridyl propargyl alcohols of formula VII.

The activation of the alcohol of formula VII is carried out e.g. by sulfonylation or halogenation. The sulfonylation of the alcohol of formula VII is a standard reaction and can be carried out e.g. with a sulfonic acid chloride, for example mesyl chloride or para-toluenesulfonic acid chloride (p-TosCl), in the presence of a tertiary amine, for example triethylamine, or an aromatic amine, for example pyridine, in a solvent, e.g. a chlorinated hydrocarbon, for example carbon tetrachloride or methylene chloride, or an amine, for example pyridine. Such reactions are generally known and are described, for example, in J. Org. Chem. 1997 (62), 8987; J. Het. Chem. 1995 (32), 875-882; and Tetrahedron Lett. 1997 (38), 8671-8674.

The halogenation of the alcohol of formula VII can be carried out analogously to standard procedures. For example, the bromination is carried out with carbon tetrabromide in the presence of triphenylphosphine (Synthesis 1998, 1015-1018) in methylene chloride. The chlorination is carried out with mineral acids, for example with concentrated hydrochloric acid (J. Org. Chem. 1955 (20), 95) or with para-toluenesulfonic acid chloride in the presence of an amine, for example triethylamine in a solvent, for example methylene chloride (Tetrahedron Lett. 1984 (25), 2295).

The preparation of the pyridyl-propynyloxy-pyridines of formula I can be carried out analogously to J. Org. Chem. 61, 4258-4261 (1996); or J. Chem. Soc., Perkin Trans I, 1979, 2756-2761 by means of etherification of the hydroxypyridines of formula II in the presence of the tosylate or mesylate or halide of formula VIII (variant a) in Scheme 2). A further method of preparing the desired target compounds of formula I is direct reaction of the propargyl alcohol of formula VII with the hydroxypyridine of formula II according to the Mitsunobu reaction in the presence of azodicarboxylic acid diethyl ester (DEAD), triphenylphosphine and a solvent such as, for example, an ether, e.g. diethyl ether or tetrahydrofuran (THF) (variant b) in Scheme 2). Etherification reactions according to Mitsunobu are described, for example, in Tetrahedron Lett. 35, 2819-2822 (1994). Suitable solvents are dimethylformamide and acetonitrile, and suitable bases are especially potassium carbonate and 1,8-diazabicyclo[5.4.0]undec-7-ene (DBU). 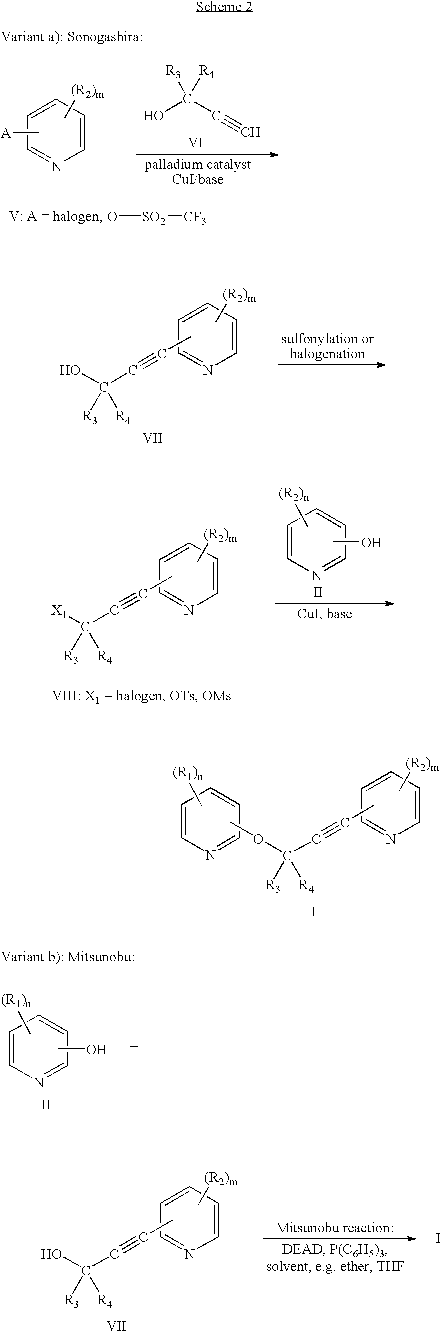
Compounds of formula I can also be obtained by further methods (see Scheme 3).

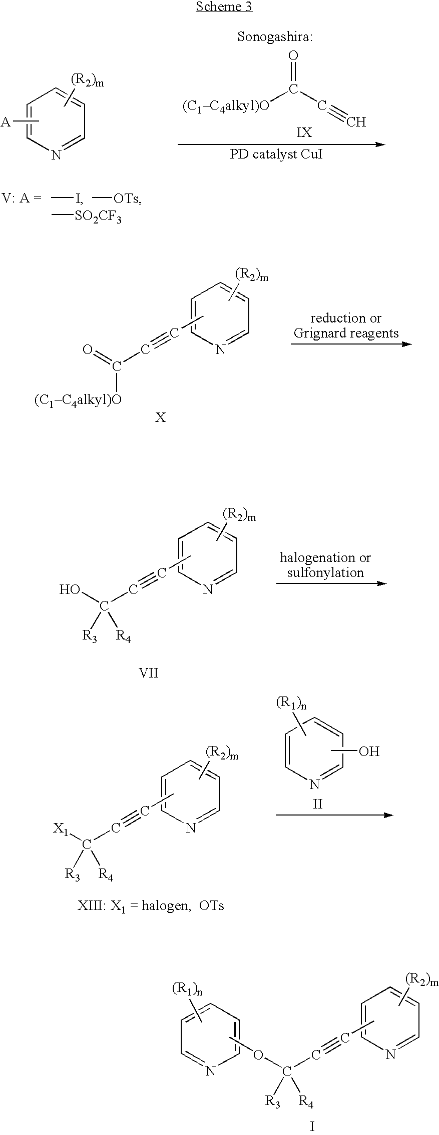

Pyridylacetylene esters of formula X can be obtained by means of Sonogashira coupling, starting from the compounds of formula IX and activated pyridine derivatives of formula V, analogously to Synthetic Communic. 1998 (28), 327-335. The esters of formula X can then be reduced or reacted with organometallic compounds, for example Grignard reagents, to form the alcohols of formula VII.

The reduction of the acetylene esters of formula X to the alcohols of formula VII can be carried out especially with hydrides by standard methods, for example with lithium aluminium hydride or sodium borohydride in a solvent, e.g. an ether, for example diethyl ether, dioxane or tetrahydrofuran, or an alcohol, for example methanol or ethanol. Such reductions are described, for example, in C. Ferri, “Reaktionen der organischen Synthese” 1978, pages 98-102.

Reactions of carboxylic acid esters with Grignard reagents are standard in organic synthesis chemistry and are described in detail, for example, in “Organikum” 1976, pages 617-625.

The subsequent etherification of the hydroxypyridines of formula II to form the compounds of formula I has already been described in detail in Scheme 2.

Further methods of preparing the compounds of formula I are shown in Scheme 4 (variant of Scheme 3).

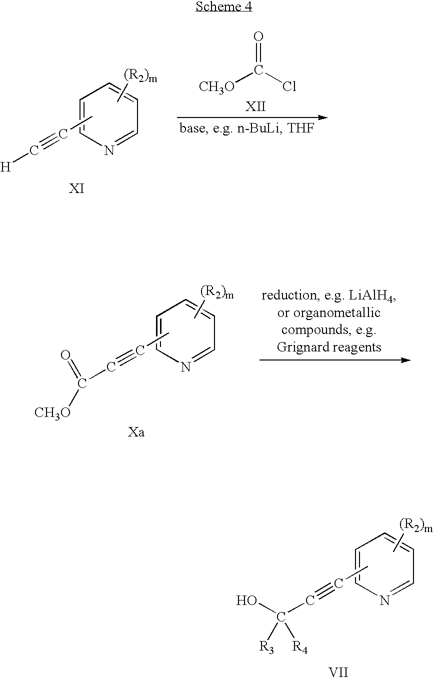

The reaction of pyridylacetylenes of formula XI with n-butyllithium (n-BuLi) and subsequent reaction with chloroformic acid methyl ester of formula XII results in the ester of formula Xa, which can be converted into the compounds of formula I entirely analogously to the method already described in Scheme 3, via an alcohol of formula VII (J. Org. Chem. 1988 (53), 4166-4171).

Compounds of formula I can also be prepared by first reacting the propargyl alcohols of formula XV with activated pyridine derivatives of formula XIV 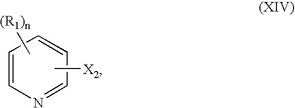
wherein X₂ is halogen, n is 1, 2, 3 or 4 and, especially in those cases where X₂ is bonded in the β- or γ-position to the ring nitrogen, R₁ is a substituent having an electron-withdrawing effect (-M and/or-I effect), e.g. —NO₂, —CN, CF₃ or COR₁₂, to form compounds of formula XVI and then in the next synthesis step carrying out a Sonogashira reaction with activated pyridine derivatives of formula V (Reaction Scheme 5).

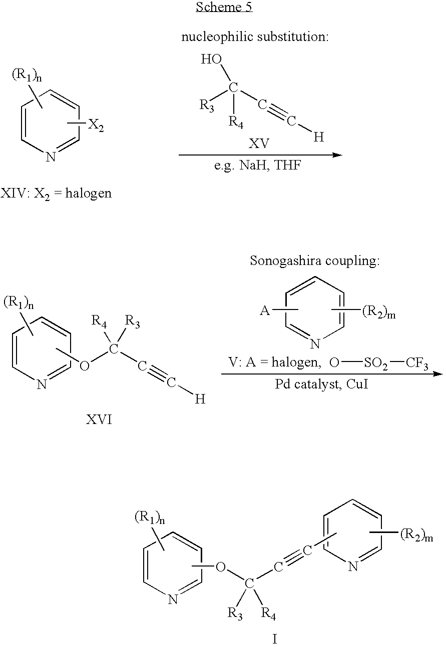

The following comments apply to the individual reaction steps (Schemes 1 to 5): The reactions to form compounds of formula I are advantageously performed in aprotic, inert organic solvents. Such solvents are hydrocarbons, such as benzene, toluene, xylene and cyclohexane, chlorinated hydrocarbons, such as dichloromethane, trichloromethane, tetra-chloromethane and chlorobenzene, ethers, such as diethyl ether, ethylene glycol dimethyl ether, diethylene glycol dimethyl ether, tetrahydrofuran and dioxane, nitriles, such as acetonitrile and propionitrile, amides, such as N,N-dimethylformamide, diethylformamide and N-methylpyrrolidinone. The reaction temperatures are preferably from −20° C. to +120° C. The reactions generally proceed slightly exothermically and can generally be carried out at room temperature. In order to shorten the reaction time or alternatively to initiate the reaction, the reaction mixture may, if appropriate, be heated to its boiling point for a short time. The reaction times may likewise be shortened by the addition of a few drops of base as reaction catalyst. Suitable bases are especially tertiary amines, such as trimethylamine, triethylamine, quinuclidine, 1,4-diazabicyclo[2.2.2]octane, 1,5-diazabicyclo[4.3.0]non-5-ene and 1,5-diaza-bicyclo[5.4.0]undec-7-ene, but it is also possible to use inorganic bases, such as hydrides, e.g. sodium or calcium hydride, hydroxides, such as sodium or potassium hydroxide, carbonates, such as sodium or potassium carbonate, or hydrogen carbonates, such as potassium or sodium hydrogen carbonate.

The compounds of formula I can be isolated in customary manner by concentration and/or evaporation of the solvent and can be purified by recrystallisation or trituration of the solid residue in solvents in which they are not readily soluble, such as ethers, aromatic hydro-carbons or chlorinated hydrocarbons.

The reagents of formulae II, III, V, VI, IX, XI, XII, XIV and XV used in Reaction Schemes 1 to 5 are known or can be prepared analogously to known methods. For example, the halogenated pyridine derivatives of formulae V and XIV can be obtained in analogous manner to that described in U.S. Pat. No. 5,468,863, and the subsequent cyanomethylation of those bromopyridines is carried out, for example, by means of nucleophilic substitution using lithium acetonitriles in analogous manner to that described in Synlett 2000(10), 1488-1490. Pyridylacetylene derivatives of formula XI and their preparation are described, for example, in Tetrahedron Organic Chemistry 20, 209-231 (2000).

For the use according to the invention of the compounds of formula I, or of compositions comprising them, there come into consideration all methods of application customary in agriculture, for example pre-emergence application, post-emergence application and seed dressing, and also various methods and techniques such as, for example, the controlled release of active ingredient. For that purpose a solution of the active ingredient is applied to mineral granule carriers or polymerised granules (urea/formaldehyde) and dried. If required, it is also possible to apply a coating (coated granules), which allows the active ingredient to be released in metered amounts over a specific period of time.

The compounds of formula I may be used as herbicides in their unmodified form, that is to say as obtained in the synthesis, but they are preferably formulated in customary manner together with the adjuvants conventionally employed in formulation technology, for example into emulsifiable concentrates, directly sprayable or dilutable solutions, dilute emulsions, wettable powders, soluble powders, dusts, granules or microcapsules. Such formulations are described, for example, on pages 9 to 13 of WO 97/34485. As with the nature of the compositions, the methods of application, such as spraying, atomising, dusting, wetting, scattering or pouring, are chosen in accordance with the intended objectives and the prevailing circumstances.

The formulations, that is to say the compositions, preparations or mixtures comprising the compound (active ingredient) of formula I or at least one compound of formula I and, usually, one or more solid or liquid formulation adjuvants, are prepared in known manner, e.g. by homogeneously mixing and/or grinding the active ingredients with the formulation adjuvants, for example solvents or solid carriers. Surface-active compounds (surfactants) may also be used in addition in the preparation of the formulations. Examples of solvents and solid carriers are given, for example, on page 6 of WO 97/34485.

Depending upon the nature of the compound of formula I to be formulated, suitable surface-active compounds are non-ionic, cationic and/or anionic surfactants and surfactant mixtures having good emulsifying, dispersing and wetting properties. Examples of suitable anionic, non-ionic and cationic surfactants are listed, for example, on pages 7 and 8 of WO 97/34485. In addition, the surfactants conventionally employed in formulation technology, which are described, inter alia, in “McCutcheon's Detergents and Emulsifiers Annual” MC Publishing Corp., Ridgewood N.J., 1981, Stache, H., “Tensid-Taschenbuch”, Carl Hanser Verlag, Munich/Vienna 1981, and M. and J. Ash, “Encyclopedia of Surfactants”, Vol. I-II, Chemical Publishing Co., New York, 1980-81, are also suitable for the preparation of the herbicidal compositions according to the invention.

The herbicidal formulations generally contain from 0.1 to 99% by weight, especially from 0.1 to 95% by weight, of herbicide, from 1 to 99.9% by weight, especially from 5 to 99.8% by weight, of a solid or liquid formulation adjuvant, and from 0 to 25% by weight, especially from 0.1 to 25% by weight, of a surfactant. Whereas commercial products will preferably be formulated as concentrates, the end user will normally employ dilute formulations. The compositions may also comprise further ingredients, such as stabilisers, for example vegetable oils or epoxidised vegetable oils (epoxidised coconut oil, rapeseed oil or soybean oil), anti-foams, for example silicone oil, preservatives, viscosity regulators, binders, tackifiers, and also fertilisers or other active ingredients.

The compounds of formula I are generally applied to plants or the locus thereof at rates of application of from 0.001 to 4 kg/ha, especially from 0.005 to 2 kg/ha. The concentration required to achieve the desired effect can be determined by experiment. It is dependent on the nature of the action, the stage of development of the cultivated plant and of the weed and on the application (place, time, method) and may vary within wide limits as a function of those parameters.

The compounds of formula I are distinguished by herbicidal and growth-inhibiting properties, allowing them to be used in crops of useful plants, especially cereals, cotton, soybeans, sugar beet, sugar cane, plantation crops, rape, maize and rice, and also for non-selective weed control. The term “crops” is to be understood as including also crops that have been made tolerant to herbicides or classes of herbicides as a result of conventional methods of breeding or genetic techniques. The weeds to be controlled may be either monocotyl-edonous or dicotyledonous weeds, such as, for example, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum halepense, Rottboellia, Cyperus, Abutilon, Sida, Xanthium, Amaranthus, Chenopodium, lpomoea, Chrysanthemum, Galium, Viola and Veronica.

The following Examples further illustrate but do not limit the invention.

### PREPARATION EXAMPLES

### Example P1

### Preparation of 2-chloro-5-iodo-pyridine

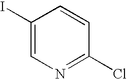

22.0 g (0.1 mol) of 2-hydroxy-5-iodo-pyridine, together with 31.0 g (0.2 mol) of phosphorus oxytrichloride, are dissolved in 100 ml of toluene at 20° C. The solution is then heated for 1 hour at reflux temperature. After the reaction is complete, excess phosphorus oxytrichloride is distilled off together with toluene, and the residue is taken up in ether. The ethereal solution is filtered over silica gel. After concentration by evaporation, 14 g of the desired title compound 2-chloro-5-iodo-pyridine are obtained in the form of light-yellow crystals having a melting point of 94-95° C.

¹H-NMR (CDCl₃): δ (ppm)=7.1-7.2 (d); 7.9-8.0 (d×d); 8.55-8.65 (d).

### Example P2

### Preparation of 5-chloro-3-fluoro-2-(prop-2-ynyloxy)-pyridine

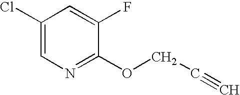

8.0 g (0.167 mol) of sodium hydride (NaH; 55%) are suspended in 200 ml of absolute tetrahydrofuran (THF) under a nitrogen atmosphere. Then, within the course of about 10 minutes, 9.9 ml (0.167 mol) of propargyl alcohol dissolved in 10 ml of absolute THF are added dropwise at a temperature of 0° C.; the ice cooling is then removed and stirring is carried out at room temperature for 45 minutes, until the evolution of gas has ceased. Then 25.0 g (0.167 mol) of 5-chloro-2,3-difluoropyridine dissolved in 50 ml of THF are added dropwise at 20-30° C., with stirring and ice cooling. Stirring at room temperature is then carried out for a further 3 hours, until gas chromatography indicates that the conversion is complete. The reaction mixture is then poured cautiously into 250 ml of water and extracted a total of three times with ethyl acetate. After separating off the organic phase, drying over sodium sulfate and filtration, concentration by evaporation is carried out; the yellow residue is purified by chromatography (eluant: ethyl acetate/hexane 1/4). 19.1 g of the desired title compound 5-chloro-3fluoro-2-(prop-2-ynyloxy)-pyridine are obtained in the form of a colourless oil.

### Example P3

### Preparation of 3,5-difluoro-2-(prop-2-ynyloxy)-pyridine

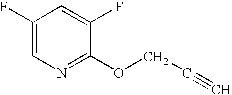

3.3 g (75.1 mmol) of NaH (55%) are suspended in 90 ml of absolute THF under a nitrogen atmosphere. Then, within the course of about 10 minutes, 4.4 ml (75.1 mmol) of propargyl alcohol dissolved in 10 ml of absolute THF are added dropwise at a temperature of 0° C.; the ice cooling is then removed and stirring is subsequently carried out at room temperature until the evolution of gas has ceased. 10.0 g (75.1 mmol) of 2,3,5-trifluoropyridine are then added at 20° C., with stirring, and stirring is carried out for a further 5 hours at room temperature until gas chromatography indicates that the reaction is complete. The reaction mixture is then poured cautiously into water and extracted with ethyl acetate; the organic phase is separated off and is washed twice with water and once with brine (saturated). The crude product obtained is dried over sodium sulfate and is then purified by column chromatography over silica gel (eluant: ethyl acetate/hexane (1/12). 6.6 g of the desired title compound 3,5-difluoro-2-(prop-2-ynyloxy)-pyridine are obtained in the form of a colourless oil. ¹H-NMR (CDCl₃): δ (ppm)=2.25 (t); 4.8 (d); 7.0 (m); 7.6 (d).

### Example P4

### Preparation of 5-fluoro-3-methoxy-2-(prop-2-ynyloxy)-pyridine

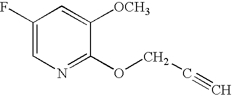

14.4 g (85.1 mmol) of 3,5-difluoro-2-(prop-2-ynyloxy)-pyridine (Example P3) are introduced into 100 ml of THF under a nitrogen atmosphere. Then, within the course of about 5 minutes, 15.8 ml (85.1 mmol) of a 30% solution (5.4M) of sodium methanolate in methanol are added dropwise at room temperature, and the reaction mixture is heated at reflux for 4 hours. The reaction mixture is cooled to room temperature and water is cautiously added. The mixture is then extracted three times with ethyl acetate; the organic phase is separated off, washed with water and brine (saturated), dried over sodium sulfate and concentrated by evaporation. The residue obtained is purified by column chromatography (eluant: ethyl acetate/hexane 1/20). 10.1 g of 5-fluoro-3-methoxy-2-(prop-2-ynyloxy)-pyridine are obtained in the form of colourless crystals having a melting point of 67-69° C.

¹H-NMR (CDCl₃): δ (ppm)=2.2 (t); 3.65 (s); 4.8 (s); 6.68 (d×d); 7.39 (d).

### Example P5

### Preparation of 5-chloro-3-methoxy-2-(prop-2-ynyloxy)-pyridine

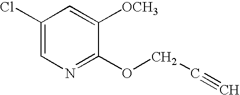

1.0 g (5.4 mmol) of 5-chloro-3-fluoro-2-(prop-2-ynyloxy)-pyridine (Example P2) is introduced into 15 ml of methanol under a nitrogen atmosphere. Then, within the course of about 5 minutes, 2.0 ml (10.8 mmol) of a 30% solution of sodium methanolate in methanol are added dropwise at room temperature; the reaction mixture is then heated to reflux temperature and stirred at that temperature for a further 18 hours. The reaction mixture is cooled to room temperature and 30 ml of water are cautiously added; the mixture is extracted three times with ethyl acetate. After being separated off, the organic phase is dried over sodium sulfate, filtered and concentrated by evaporation. The yellowish residue obtained is purified by chromatography (eluant: ethyl acetate/hexane 1/4). 0.65 g of the desired title compound 5-chloro-3-methoxy-2-(prop-2-ynyloxy)-pyridine is obtained in the form of colourless crystals having a melting point of 62-64° C.

¹H-NMR (CDCl₃): δ (ppm)=2.45 (t); 3.85 (s); 5.0 (s); 7.05 (d); 7.7 (d).

### Example P6

### Preparation of 2-[3-(6-chloro-pyrid-3-yl)-prop-2-ynyloxy]-5-fluoro-3-methoxy-pyridine

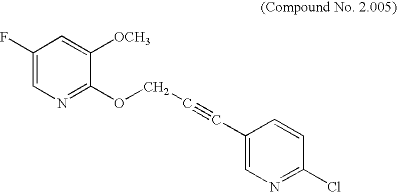

2.4 g (10.0 mmol) of 2-chloro-5-iodo-pyridine (Example P1), 1.8 g (10.06 mmol) of 5-fluoro-3-methoxy-2-(prop-2-ynyloxy)-pyridine (Example P4) and 0.58 g (0.5 mmol) of tetrakis-(triphenylphosphine)-palladium (Pd(PPh₃)₄) are dissolved at a temperature of 20° C. in 20 ml of piperidine under an argon atmosphere. After stirring for 5 minutes, 164 mg (0.86 mmol) of copper(I) iodide (CuI) are added, whereupon the temperature rises momentarily to about 55° C. After stirring for a further 2 hours at a temperature of 20° C., the reaction mixture is poured onto 40 ml of saturated ammonium chloride solution and, after stirring briefly, is extracted with diethyl ether; the organic phase is separated off and dried over magnesium sulfate. The crude product is subjected to flash chromatography over silica gel using ethyl acetate/hexane (1/10 to 1/4) as gradient eluant. 2.1 g of the desired target compound 2-[3-(6chloro-pyrid-3-yl)-prop-2-ynyloxy]-5-fluoro-3-methoxy-pyridine are obtained in the form of white crystals having a melting point of 101-102° C.

¹H-NMR (CDCl₃): δ (ppm)=3.9 (s); 5.25 (s); 6.95 (d×d); 7.3 (d); 7.66 (d); 7.7 (d×d); 8.46(d).

### Example P7

### Preparation of 5-chloro-2-[3-(6-chloro-pyrid-3-yl)-prop-2-ynyloxy]-3-methoxy-pyridine

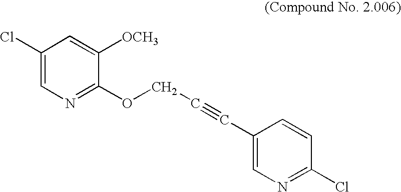

2.4 g (10.0 mmol) of 2-chloro-5-iodo-pyridine (Example P1), 1.99 g (10.06 mmol) of 5-chloro-3-methoxy-2-(prop-2-ynyloxy)-pyridine (Example P5) and 0.58 g (0.5 mmol) of Pd(PPh₃)₄ are dissolved at a temperature of 20° C. in 20 ml of piperidine under an argon atmosphere. After stirring for 5 minutes, 164 mg (0.86 mmol) of CuI are added, whereupon the temperature rises momentarily to about 40° C. and a precipitate forms. After stirring for a further 2 hours at a temperature of 20° C., the reaction mixture is poured onto 40 ml of saturated ammonium chloride solution and, after stirring for half an hour, is extracted with diethyl ether, and the organic phase is separated off. The organic phase is then washed twice with saturated ammonium chloride solution and brine and is dried over sodium sulfate. The crude product is chromatographed over silica gel using ethyl acetate/hexane as eluant. 1.4 g of the desired target compound 5chloro-2-[3-(6-chloro-pyrid-3-yl-prop-2-ynyloxy)-3-methoxy-pyridine are obtained in the form of white crystals having a melting point of 100-101° C.

¹H-NMR (CDCl₃): δ (ppm)=4.05 (s); 5.4 (s); 7.25 (d); 7.45 (d); 7.83 (d×d); 7.87 (d); 8.6(d).

### Example P8

### Preparation of 2-[3-(6-cyanomethoxy-pyrid-2-yl)-prop-2-ynyloxy]-5-chloro-3-methoxypyridine

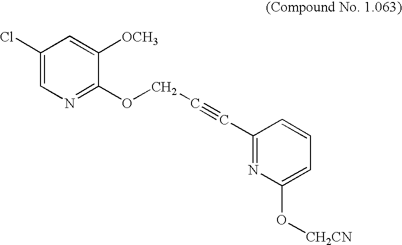

5.63 ml of tetrabutylammonium fluoride (1 M solution in THF) are added to a solution of 600 mg (2.82 mmol) of [(6-bromopyrid-2-yl)oxy]acetonitrile (Example P10), 557 mg (4.22 mmol) of 5-chloro-3-methoxy-2-(prop-2-ynyloxy)-pyridine (Example P5), 107 mg (0.56 mmol) of copper(I) iodide and 198 mg (0.282 mmol) of bis-triphenylphosphine-palladium dichloride (Pd(PPh₃)₂Cl₂) in 15 ml of dioxane. The resulting reaction mixture is stirred for 6 hours at 50° C. under an argon atmosphere and is then allowed to cool to 20° C. The solvent is removed under reduced pressure and the crude product obtained is purified by means of flash chromatography (eluant: ethyl acetate/petroleum ether 1/3). The desired title compound is obtained, in a yield of 390 mg (42% of theory), in the form of a yellow solid having a melting point of 118° C.

R_{f}=0.50 in ethyl acetate/petroleum ether 1/3;

¹H-NMR (CDCl₃): δ(ppm)=3.89 (s, 3H); 5.04 (s, 2H); 5.26 (s, 2H); 6.80 (d, J=8.5 Hz, 1H); 7.07 (d, J=1.9 Hz, 1H); 7.16 (d, J=7.6 Hz, 1H); 7.60 (d×d, J=7.6 and 8.5 Hz, 1H); 7.71 (d, J=2.2 Hz, 1H).

### Example P9

### Preparation of 2-hydroxy-6-bromopyridine

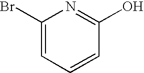

4.67 ml of sulfuric acid 98% are added to a solution of 4.0 9 (23.12 mmol) of 6-bromo-2-aminopyridine in 67 ml of water at 0-5° C. The reaction temperature is maintained at 0-5° C. and 1.75 g (25.43 mmol) of sodium nitrite in 15 ml of water are added dropwise. After being left to stand at 0° C. for 30 minutes, the mixture is added to an aqueous solution of 23.12 g (92.48 mmol in 40 ml of water) of copper sulfate (CuSO_{4·5} H₂O) and heated to 100° C. The mixture is maintained at that temperature for 1.5 hours, is then cooled to 20° C. and extracted with dichloromethane. The organic phase is separated off and dried over magnesium sulfate, the drying medium is filtered off and the solvent is removed under reduced pressure. The title compound is obtained, in a yield of 3.476 g (86% of theory), in the form of a yellow solid having a melting point of 110° C.

R_{f}=0.06 in ethyl acetate/petroleum ether 1/1;

¹H-NMR (CDCl₃): δ(ppm)=6.71 (d, J=8.5 Hz, 1H); 6.83 (d, J=7.5 Hz, 1H); 7.44 (d×d, J=7.5 and 8.5 Hz, 1H).

### Example P10

### Preparation of [(6-bromopyrid-2-yl)oxy]acetonitrile

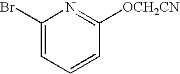

1.9 g (13.79 mmol) of potassium carbonate and 0.57 ml (0.67 g, 8.96 mmol) of chloro-acetonitrile are added to a solution of 1.2 g (6.9 mmol) of 6-bromopyridin-2-ol (Example P9) in 10 ml of 1-methyl-2-pyrrolidone (NMP). The reaction mixture is stirred for 2.5 hours at 20° C. under argon gas. The solvent is removed under reduced pressure and the crude product obtained is purified by means of flash chromatography (eluant: ethyl acetate/petroleum ether 1/3). The desired title compound is obtained in a yield of 1.13 g (77% of theory).

R_{f}=0.52 in ethyl acetate/petroleum ether 1/3;

¹H-NMR (CDCl₃): δ(ppm)=5.03 (s, 2H); 6.80 (d, J=8.2 Hz, 1H); 7.19 (d, J=7.5 Hz, 1H); 7.52 phosphine-palladium (d×d, J=7.5 and 8.2 Hz, 1H).

¹³C-NMR (CDCl₃): δ (ppm)=50.67 (OCH₂), 109.48; 122.35 and 141.38 (arom. CH).

### Example P11

### Preparation of 4-chloro-2-iodopyridine (Eur. J. Org. Chem. 2001, 603-606)

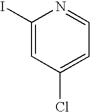

Commercial 4-chloropyridine hydrochloride is neutralised by adding potassium carbonate and is then extracted with dichloromethane.

A solution of 4.74 g (53.2 mmol) of 2-(dimethylamino)-ethanol in 30 ml of THF is cooled to −5° C., and 66.5 ml (106.4 mmol) of n-butyllithium are added dropwise under an argon atmosphere. The temperature is maintained at 0° C. for 30 minutes and is then cooled to −78° C., and a solution of 1.5 g (13.3 mmol) of 4-chloropyridine in 30 ml of THF is added dropwise. Stirring is carried out at −78° C. for 1 hour, and a solution of 16.9 g (66.5 mmol) of iodine in 30 ml of THF is added dropwise to the mixture. After 30 minutes at −78° C., the reaction mixture is allowed to warm slowly, within the course of 1 hour, up to 20° C. and, at 0° C., is hydrolysed with 60 ml of water. The aqueous phase is extracted with dichloro-methane, and the combined organic phases are washed with a solution of sodium bisulfite (Na₂S₂O₃) and dried over magnesium sulfate; the solvent is concentrated under reduced pressure. The desired title compound is obtained, in a yield of 1.72 g (54% of theory), in the form of a brown oil.

¹H-NMR (CDCl₃): δ(ppm)=7.30 (d×d, J=1.5 and 5.3 Hz, 1H); 7.77 (d, J=1.5 Hz, 1H); 8.26 (d, J=5.3 Hz, 1H).

### Example P12

### Preparation of 2-[3-(4-chloro-pyrid-2-yl)-prop-2-ynyloxy]-5-chloro-3-methoxypyridine

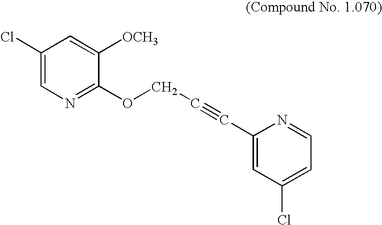

2.1 ml of tetrabutylammonium fluoride (1M solution in THF) is added to a solution of 250 mg (1.04 mmol) of 4-chloro-2-iodopyridine (Example P11), 309 mg (1.56 mmol) of 5-chloro-3-methoxy-2-(prop-2-ynyloxy)-pyridine (Example P5), 40 mg (0.21 mmol) of copper(I) iodide and 74 mg (0.105 mmol) of bis-triphenylphosphine-palladium dichloride (Pd(PPh₃)₂Cl₂) in 7 ml of dioxane. The resulting reaction mixture is stirred at 50° C. under an argon atmosphere for 4 hours and is then allowed to cool to 20° C. The solvent is removed under reduced pressure and the crude product obtained is purified by means of flash chromatography (eluant: ethyl acetate/petroleum ether 1/2). The desired title compound is obtained, in a yield of 205 mg (63% of theory), in the form of a beige solid having a melting point of 129° C.

R_{f}=0.38 in ethyl acetate/petroleum ether 1/2;

¹H-NMR (CDCl₃): δ(ppm)=3.88 (s, 3H); 5.25 (s, 2H); 7.06 (d, J=1.9 Hz, 1H); 7.24 (d×d), J=2.2 and 5.3 Hz,1H); 7.44 (d, J=2.2 Hz, 1H); 7.70 (d, J=1.9 Hz,1H); 8.45 (d, J=5.3 Hz, 1H).

### Example P13

### Preparation of 2-[3-(6-chloro-pyrid-3-yl)-prop-2-ynyloxy]-5-chloro-3-methoxypyridine

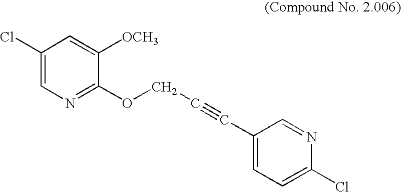

4.81 ml of tetrabutylammonium fluoride (1M solution in THF) is added to a solution of 500 mg (2.08 mmol) of commercial 2-chloro-5-iodopyridine, 619 mg (3.13 mmol) of 5-chloro-3-methoxy-2-(prop-2-ynyloxy)-pyridine (Example P5), 79 mg (0.42 mmol) of copper(I) iodide and 147 mg (0.21 mmol) of bis-triphenylphosphine-palladium dichloride (Pd(PPh₃)₂Cl₂) in 14 ml of dioxane. The resulting reaction mixture is stirred at 50° C. under an argon atmosphere for 4 hours and is then allowed to cool to 20° C. The solvent is removed under reduced pressure and the crude product obtained is purified by means of flash chromatography (eluant: ethyl acetate/petroleum ether 1/2). The desired title compound is obtained, in a yield of 285 mg (44% of theory), in the form of a yellow solid having a melting point of 94° C.

R_{f}=0.58 in ethyl acetate/petroleum ether 1/2;

¹H-NMR (CDCl₃): δ(ppm)=3.89 (s, 3H); 5.24 (s, 2H); 7.07 (d, J=2.1 Hz, 1H); 7.26 (d×d, J=0.7 and 4.1 Hz, 1H); 7.66 (d×d, J=1.9 and 4.1 Hz, 1H); 7.70 (d, J=2.1 Hz, 1H); 8.45 (d, J=1.9 Hz, 1H).

### Example P14

### Preparation of 2-[2-(4-methyl-pyrid-2-yl)-prop-2-ynyloxy]-5-chloro-3-methoxypyridine

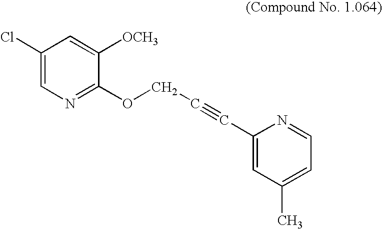

3.5 ml of tetrabutylammonium fluoride (1M solution in THF) is added to a solution of 300 mg (1.74 mmol) of commercial 2-bromo-4-methylpyridine, 517 mg (2.62 mmol) of 5-chloro-3-methoxy-2-(prop-2-ynyloxy)-pyridine (Example P5), 66 mg (0.35 mmol) of copper(I) iodide and 122 mg (0.17 mmol) of bis-triphenylphosphine-palladium dichloride (Pd(PPh₃)₂Cl₂) in 12 ml of dioxane. The resulting reaction mixture is stirred at 50° C. under an argon atmosphere for 16 hours and is then allowed to cool to 20° C. The solvent is removed under reduced pressure and the crude product obtained is purified by means of flash chromatography (eluant: ethyl acetate/petroleum ether 1/2). The desired title compound is obtained in a yield of 235 mg (47% of theory), having a melting point of 106-108° C.

R_{f}=0.27 in ethyl acetate/petroleum ether 1/2;

¹H-NMR (CDCl₃): δ(ppm)=2.29 (s, 3H); 3.85 (s, 3H); 5.23 (s, 2H); 7.03 (d, J=2.1 Hz, 1H); 7.04 (d, J=4.1H); 7.26 (s,1H); 7.68 (d, J=2.1 Hz,1H); 8.38 (d, J=4.1 Hz,1H).

### Example P15

### Preparation of 2-[2-(5-methyl-pyrid-2-yl)-prop-2-ynyloxy]-5-chloro-3-methoxypyridine

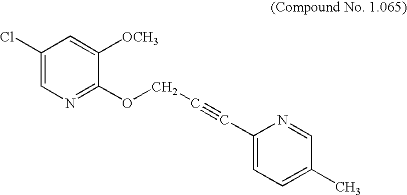

3.5 ml of tetrabutylammonium fluoride (1M solution in THF) are added to a solution of 300 mg (1.74 mmol) of commercial 2-bromo-5-methylpyridine, 517 mg (2.62 mmol) of 5-chloro-3-methoxy-2-(prop-2-ynyloxy)-pyridine (Example P5), 66 mg (0.35 mmol) of copper(I) iodide and 122 mg (0.17 mmol) of bis-triphenylphosphine-palladium dichloride (Pd(PPh₃)₂Cl₂) in 12 ml of dioxane. The resulting reaction mixture is stirred at 50° C. under an argon atmosphere for 5 hours and is then allowed to cool to 20° C. The solvent is removed under reduced pressure and the crude product obtained is purified by means of flash chromatography (eluant: ethyl acetate/petroleum ether 1/2). The desired title compound is obtained, in a yield of 275 mg (55% of theory), in the form of a beige solid having a melting point of 113° C.

R_{f}=0.25 in ethyl acetate/petroleum ether 1/2;

¹H-NMR (CDCl₃): δ(ppm)=2.32 (s, 3H); 3.87 (s, 3H); 5.24 (s, 2H); 7.04 (d, J=1.9 H); 7.32 (d, J=7.9 Hz, 1H); 7.40 (d, J=7.9 Hz, 1H); 7.70 (d, J=1.9 Hz, 1H); 8.38 (s, 1H).

### Example P16

### Preparation of 2-[2-(5-trifluoromethyl-pyrid-2-yl)-prop-2-ynyloxy]-5-chloro-3-methoxypyridine

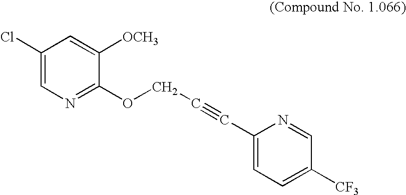

4.5 ml of tetrabutylammonium fluoride (1M solution in THF) is added to a solution of 500 mg (2.21 mmol) of commercial 2-bromo-5-trifluoromethylpyridine, 655 mg (3.32 mmol) of 5-chloro-3-methoxy-2-(prop-2-ynyloxy)-pyridine (Example P5), 84 mg (0.44 mmol) of copper(I) iodide and 155 mg (0.22 mmol) of bis-triphenylphosphine-palladium dichloride (Pd(PPh₃)₂Cl₂) in 15 ml of dioxane. The resulting reaction mixture is stirred at 50° C. under an argon atmosphere for 4 hours and is then allowed to cool to 20° C. The solvent is removed under reduced pressure and the crude product obtained is purified by means of flash chromatography (eluant: ethyl acetate/petroleum ether 1/2). The desired title compound is obtained, in a yield of 450 mg (59% of theory), in the form of a beige solid having a melting point of 114° C.

R_{f}=0.48 in ethyl acetate/petroleum ether 1/2;

¹H-NMR (CDCl₃): δ(ppm)=3.88 (s, 3H); 5.27 (s, 2H); 7.06 (d, J=2.1 Hz, 1H); 7.55 (d, J=8.1 Hz,1H); 7.85 (d×d, J=8.1 and 1.5 Hz, 1H); 7.70 (d, J=2.1 Hz,1H); 8.82 (s, 1H).

### Example P17

### Preparation of 2-[2-(6-methoxy-pyrid-2-yl)-prop-2-ynyloxy]-5-chloro-3-methoxypyridine

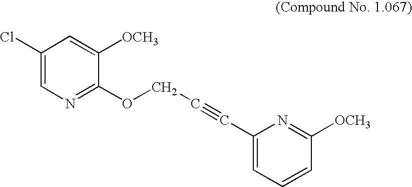

5.3 ml of tetrabutylammonium fluoride (1M solution in THF), 500 mg (2.66 mmol) of 2-bromo-6-methoxypyridine, 788 mg (3.99 mmol) of 5-chloro-3-methoxy-2-(prop-2-ynyloxy)-pyridine (Example P5), 101 mg (0.53 mmol) of copper(I) iodide and 187 mg (0.26 mmol) of bis-triphenylphosphine-palladium dichloride (Pd(PPh₃)₂Cl₂) in 18 ml of dioxane are reacted in analogous manner to that described in the previous Examples and are then worked up and purified. The desired title compound is obtained, in a yield of 385 mg (48% of theory) in the form of a white solid having a melting point of 116° C.

R_{f}=0.52 in ethyl acetate/petroleum ether 1/2;

¹H-NMR (CDCl₃): δ(ppm)=3.88 (s, 3H); 3.92 (s, 3H); 5.26 (s, 2H); 6.68 (d, J=8.2 Hz, 1H); 7.04 (d, J=8.2 Hz, 1H); 7.06 (d, J=2.2 Hz, 1H); 7.48 (d, J=8.2 Hz, 1H); 7.70 (d, J=2.2 Hz, 1H).

### Example P18

### Preparation of 2-[2-(6-methyl-pyrid-2-yl)-prop-2-ynyloxy]-5-chloro-3-methoxypyridine

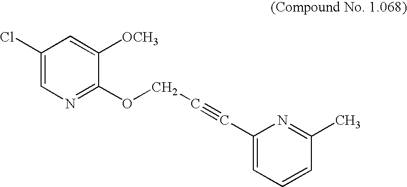

5.8 ml of tetrabutylammonium fluoride (1M solution in THF), 500 mg (2.90 mmol) of commercial 2-bromo-6-methylpyridine, 861 mg (4.36 mmol) of 5-chloro-3-methoxy-2-(prop-2-ynyloxy)-pyridine (Example P5), 110 mg (0.58 mmol) of copper(I) iodide and 204 mg (0.29 mmol) of bis-triphenylphosphine-palladium dichloride (Pd(PPh₃)₂Cl₂) in 18 ml of dioxane are reacted in analogous manner to that described in the previous Examples and are then worked up and purified. The desired title compound is obtained, in a yield of 530 mg (63% of theory), in the form of a beige solid having a melting point of 96° C.

R_{f}=0.39 in ethyl acetate/petroleum ether 1/2;

¹H-NMR (CDCl₃): δ(ppm)=2.53 (s, 3H); 3.87 (s, 3H); 5.25 (s, 2H); 7.05 (d, J=2.2 Hz, 1H); 7.08 (d, J=7.8 Hz, 1H); 7.24 (d, J=7.8 Hz, 1H); 7.50 (d, J=7.8 Hz, 1H); 7.69 (d, J=2.2 Hz, 1H).

### Example P19

### Preparation of 2-[2-(5-cyanomethylene-pyrid-2-yl)-prop-2-ynyloxy]-5-chloro-3-methoxypyridine

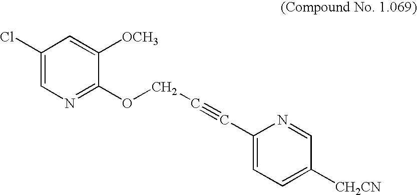

6.1 ml of tetrabutylammonium fluoride (1M solution in THF), 600 mg (3.04 mmol) of (6-bromopyrid-3-yl)acetonitrile (Example P21), 902 mg (4.56 mmol) of 5-chloro-3-methoxy-2-(prop-2-ynyloxy)-pyridine (Example P5), 116 mg (0.61 mmol) of copper(I) iodide and 214 mg (0.30 mmol) of bis-triphenylphosphine-palladium dichloride (Pd(PPh₃)₂Cl₂) in 20 ml of dioxane are reacted in analogous manner to that described in the previous Examples and are then worked up and purified by means of flash chromatography (eluant: ethyl acetate/petroleum ether 1/1). The desired title compound is obtained, in a yield of 585 mg (61% of theory), in the form of a beige solid having a melting point of 127° C.

R_{f}=0.19 in ethyl acetate/petroleum ether 1/1;

¹H-NMR (CDCl₃): δ(ppm)=3.77 (s, 2H); 3.88 (s, 3H); 5.26 (s, 2H); 7.06 (d, J=1.9 Hz, 1H); 7.47 (d, J=8.2 Hz, 1H); 7.66 (d×d, J=8.2 and 2.2 Hz, 1H); 7.70 (d, J=1.9 Hz, 1H); 8.51 (d, J=2.2 Hz, 1H).

### Example P20

### Preparation of 2-bromo-5-(bromomethyl)pyridine

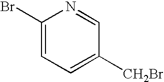

1.24 g (7.0 mmol) of N-bromosuccinimide are added to a solution of 1.0 g (5.8 mmol) of commercial 2-bromo-5-methylpyridine in 10 ml of carbon tetrachloride under argon gas. The mixture is heated to reflux temperature, and 100 mg (0.6 mmol) of azobisisobutyronitrile (AIBN) are then added. After heating at reflux temperature for 18 hours, the reaction mixture is cooled to 20° C. and filtered. The solvent is removed under reduced pressure, and the crude product obtained is purified by means of flash chromatography (eluant: ethyl acetate/petroleum ether 1/3). The desired title compound is obtained in a yield of 825 mg (56% of theory).

R_{f}=0.60 in ethyl acetate/petroleum ether 1/3;

¹H-NMR (CDCl₃): δ(ppm)=4.40 (s, 2H); 7.45 (d, J=8.0 Hz, 1H); 7.57 (d×d, J=2.5 and 8.0 Hz, 1 H); 8.36 (d, J=2.5 Hz,1H).

### Example P21

### Preparation of (6-bromopyrid-3-yl)acetonitrile

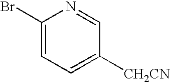

0.52 9 (8.0 mmol) of potassium cyanide is added to a solution of 0.80 g (3.2 mmol) of 2-bromo-5-(bromomethyl)pyridine (Example P20) in a mixture of 8 ml of acetonitrile/water 8/2. The resulting mixture is then heated at reflux temperature for 18 hours. After cooling to 20° C., the reaction mixture is extracted with dichloromethane, the combined organic extracts are washed with a solution of sodium bicarbonate (NaHCO₃) and dried over magnesium sulfate, and the solvent is removed under reduced pressure. The crude product obtained is purified by means of flash chromatography (eluant: ethyl acetate/petroleum ether 1/3). The desired title compound is obtained, in a yield of 390 mg (62% of theory), in the form of a yellow solid having a melting point of 64° C.

R_{f}=0.20 in ethyl acetate/petroleum ether 1/3;

¹H-NMR (CDCl₃): δ(ppm)=3.73 (s, 2H); 7.51 (d, J=8.5 Hz, 1H); 7.55 (d×d, J=2.5 and 8.5 Hz, 1H); 8.33 (d, J=2.5 Hz, 1H).

### Example P22

### Preparation of 2-[2-(4-cyanomethylene-pyrid-2-yl)-prop-2-ynyloxy]-5-chloro-3-methoxypyridine

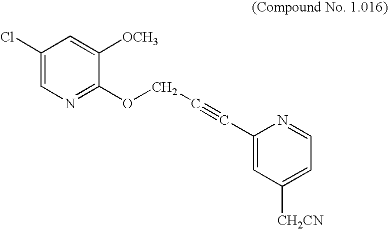

3.9 ml of tetrabutylammonium fluoride (1M solution in THF), 380 mg (1.93 mmol) of (2-bromopyrid-4-yl)acetonitrile (Example P24), 571 mg (2.89 mmol) of 5-chloro-3-methoxy-2-(prop-2-ynyloxy)-pyridine (Example P5), 73 mg (0.38 mmol) of copper(I) iodide and 135 mg (0.19 mmol) of bis-triphenylphosphine-palladium dichloride (Pd(PPh₃)₂Cl₂) in 20 ml of dioxane are reacted in analogous manner to that described in the previous Examples and are then worked up and purified by means of flash chromatography (eluant: ethyl acetate/petroleum ether 1/1). The desired title compound is obtained, in a yield of 210 mg (34% of theory), in the form of a yellow viscous mass.

R_{f}=0.27 in ethyl acetate/petroleum ether 1/1;

¹H-NMR (CDCl₃): δ(ppm)=3.47 (s, 2H); 3.86 (s, 3H); 5.24 (s, 2H); 7.05 (d, J=2.2 Hz, 1H); 7.19 (d×d, J=5.1 and 1.6 Hz, 1H); 7.40 (d, J=1.6 Hz, 1H); 7.68 (d, J=2.2 Hz, 1H); 8.54 (d, J=5.1 Hz, 1H).

### Example P23

### Preparation of 2-bromo-4-(bromomethyl)pyridine

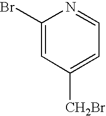

6.21 g (34.9 mmol) of N-bromosuccinimide are added to a solution of 5.0 g (29.1 mmol) of commercial 2-bromo-4-methylpyridine in 50 ml of carbon tetrachloride under argon gas. The resulting mixture is heated to reflux temperature, and 750 mg (0.9 mmol) of azobisisobutyro nitrile (AIBN) are then added in 3 portions at intervals of 4 hours. After heating for 20 hours at reflux temperature, the reaction mixture is cooled to 20° C., and the mixture is filtered. The solvent is removed under reduced pressure and the crude product obtained is purified by means of flash chromatography (eluant: ethyl acetate/petroleum ether 1/2). The desired target compound is obtained, in a yield of 1.85 g (25% of theory), in the form of a white solid.

R_{f}=0.67 in ethyl acetate/petroleum ether 1/2;

¹H-NMR (CDCl₃): δ(ppm)=4.33 (s, 2H); 7.26 (d×d, J=5.1 and 1.5 Hz,1H); 7.51 (d, J=1.5 Hz, 1H); 8.34 (d, J=5.1 Hz, 1H).

### Example P24

### Preparation of (2-bromopyrid-4-yl)acetonitrile

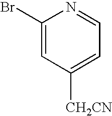

1.44 g (22.1 mmol) of potassium cyanide are added to a solution of 1.85 g (7.37 mmol) of 2-bromo-4-(bromomethyl)pyridine (Example P23) in a mixture of 20 ml of acetonitrlle/water 8/2. The resulting mixture is then heated at reflux temperature for 4 hours. After cooling to 20° C., the reaction mixture is extracted with dichloromethane, the combined organic extracts are washed with a solution of sodium bicarbonate (NaHCO₃) and dried over magnesium sulfate, and the solvent is removed under reduced pressure. The crude product obtained is purified by means of flash chromatography (eluant: ethyl acetate/petroleum ether 1/2). The desired title compound is obtained in a yield of 420 mg (29% of theory).

R_{f}=0.25 in ethyl acetate/petroleum ether 1/2;

¹H-NMR (CDCl₃): δ(ppm)=3.76 (s, 2H); 7.26 (d×d, J=5.1 and 0.7 Hz, 1H); 7.51 (d, J=0.7 Hz, 1H); 8.39 (d, J=5.1 Hz,1H).

In a manner analogous to that described in Examples P1 to P24 or in accordance with the methods as shown in Reaction Schemes 1-5 and in the references indicated, it is also possible to obtain the preferred compounds listed in the following Tables. In the column headed “Phys. data”, the temperatures indicate the melting point (m.p.) of the compounds in question.

TABLE 1
Compounds of formula I₁

| | |
|---|---|
| | (I₁) |
| 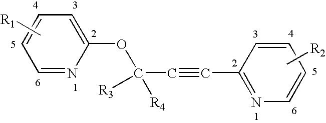 | |

| Comp. | | | | | Phys. data |
|---|---|---|---|---|---|
| No. | R₁ | R₂ | R₃ | R₄ | m.p.(° C.) |
| 1.001 | 3-F | 4-CH₂CN | H | H | |
| 1.002 | 3-Cl | 4-CH₂CN | H | H | |
| 1.003 | 3-F, 5-F | 4-Cl | H | H | |
| 1.004 | 3-F, 5-F | 4-Br | H | H | |
| 1.005 | 3-F, 5-F | 4-CH₂CN | H | H | |
| 1.006 | 3-F, 5-F | 4-CH₃ | H | H | |
| 1.007 | 3-F, 5-Cl | 4-Cl | H | H | |
| 1.008 | 3-F, 5-Cl | 4-Br | H | H | |
| 1.009 | 3-F, 5-Cl | 4-CH₂CN | H | H | |
| 1.010 | 3-F, 5-F | 4-CH(CH₃)CN | CH₃ | H | |
| 1.011 | 3-F, 5-F | 4-CH₂CN | CH₃ | CH₃ | |
| 1.012 | 3-F, 5-Cl | 4-CH(CH₃)CN | CH₃ | H | |
| 1.013 | 3-F, 5-Cl | 4-CH₂CN | CH₃ | CH₃ | |
| 1.014 | 3-F, 5-Cl | 4-CH₂CN | F | H | |
| 1.015 | 3-OCH₃, 5-F | 4-CH₂CN | H | H | |
| 1.016 | 3-OCH₃, 5-Cl | 4-CH₂CN | H | H | ¹H-NMR |
| | | | | | (Example |
| | | | | | P22) |
| 1.017 | 3-OCH₃, 5-Br | 4-CH₂CN | H | H | |
| 1.018 | 3-OCH₃, 5-CN | 4-CH₂CN | H | H | |
| 1.019 | 3-OCH₃, 5-F | 4-CH(CH₃)CN | H | H | |
| 1.020 | 3-OCH₃, 5-Cl | 4-CH(CH₃)CN | H | H | |
| 1.021 | 3-OCH₃, 5-Br | 4-CH(CH₃)CN | H | H | |
| 1.022 | 3-OCH₃, | 4-CH(CH₃)CN | H | H | |
| | 5-CH═NOCH₃ | | | | |
| 1.023 | 3-OCH₃, 5-CN | 4-CH(CH₃)CN | H | H | |
| 1.024 | 3-OCH₃, 5-F | 4-C(CH₃)₂CN | H | H | |
| 1.025 | 3-OCH₃, 5-Cl | 4-C(CH₃)₂CN | H | H | |
| 1.026 | 3-F, 5-F | 5-Cl | H | H | |
| 1.027 | 3-F, 5-F | 6-Cl | H | H | |
| 1.028 | 3-OCH₃, 5-F | 5-Cl | H | H | |
| 1.029 | 3-OCH₃, 5-F | 6-Cl | H | H | |
| 1.030 | 3-F, 5-F | 6-CH₂CN | H | H | |
| 1.031 | 3-OCH₃, 5-F | 6-CH₂CN | H | H | |
| 1.032 | 3-OCH₃, 5-Cl | 6-CH₂CN | H | H | solid |
| 1.033 | 3-OCH₃, 5-F | 6-CH(CH₃)CN | H | H | |
| 1.034 | 3-OCH₃, 5-Cl | 6-CH(CH₃)CN | H | H | |
| 1.035 | 3-OCH₃, 5-F | 4-CHF₂ | H | H | |
| 1.036 | 3-OCH₃, 5-Cl | 4-CHF₂ | H | H | |
| 1.037 | 3-OCH₃, | 4-CHF₂ | H | H | |
| | 5-CH═NOCH₃ | | | | |
| 1.038 | 3-OCH₃, 5-F | 6-CH₂CN | CH₃ | CH₃ | |
| 1.039 | 3-OCH₃, 5-Cl | 6-CH₂CN | CH₃ | CH₃ | |
| 1.040 | 3-F, 5-F | 6-CH₂CN | F | F | |
| 1.041 | 3-F, 5-Cl | 6-CH₂CN | F | F | |
| 1.042 | 3-OCH₃, 5-F | 6-CF₃ | H | H | |
| 1.043 | 3-OCH₃, 5-Cl | 6-CF₃ | H | H | |
| 1.044 | 3-OCH₃, | 6-CF₃ | H | H | |
| | 5-CH═NOCH₃ | | | | |
| 1.045 | 3-F, 5-F | 6-NHCOCH₃ | H | H | |
| 1.046 | 3-OCH₃, 5-F | 6-NHCOCH₃ | H | H | |
| 1.047 | 3-OCH₃, 5-Cl | 6-NHCOCH₃ | H | H | |
| 1.048 | 3-OCH₃, 5-F | 6-NHSO₂CH₃ | H | H | |
| 1.049 | 3-OCH₃, 5-Cl | 6-NHSO₂CH₃ | H | H | |
| 1.050 | 3-OCH₃, 5-F | 6-CHF₂ | H | H | |
| 1.051 | 3-OCH₃, 5-Cl | 6-CHF₂ | H | H | |
| 1.052 | 3-F, 5-Cl | 5-Cl | OCH₃ | H | |
| 1.053 | 3-F, 5-Cl | 5-Cl | CN | H | |
| 1.054 | 3-OCH₃, 5-F | 3-Cl | H | H | |
| 1.055 | 3-OCH₃, 5-F | 3-Br | H | H | |
| 1.056 | 3-F, 5-F | 4-NO₂ | H | H | |
| 1.057 | 3-OCH₃, 5-F | 4-NO₂ | H | H | |
| 1.058 | 3-F, 5-CF₃ | 4-CH₂CN | H | H | |
| 1.059 | 3-Cl, 5-CF₃ | 4-CH₂CN | H | H | |
| 1.060 | 3-F, 5-CF₃ | 4-CH(CH₃)CN | H | H | |
| 1.061 | 3-Cl, 5-CF₃ | 4-CH(CH₃)CN | H | H | |
| 1.062 | 3-OCH₃, 5-CF₃ | 6-CH₂CN | H | H | |
| 1.063 | 3-OCH₃, 5-Cl | 6-OCH₂CN | H | H | 118; |
| | | | | | ¹H-NMR |
| | | | | | (Example |
| | | | | | P8) |
| 1.064 | 3-OCH₃, 5-Cl | 4-CH₃ | H | H | 106-108; |
| | | | | | ¹H-NMR |
| | | | | | (Example |
| | | | | | P14) |
| 1.065 | 3-OCH₃, 5-Cl | 5-CH₃ | H | H | 113 |
| 1.066 | 3-OCH₃, 5-Cl | 5-CF₃ | H | H | 114 |
| 1.067 | 3-OCH₃, 5-Cl | 6-OCH₃ | H | H | 116 |
| 1.068 | 3-OCH₃, 5-Cl | 6-CH₃ | H | H | 96 |
| 1.069 | 3-OCH₃, 5-Cl | 5-CH₂CN | H | H | 127 |
| 1.070 | 3-OCH₃, 5-Cl | 4-Cl | H | H | 129 |
| 1.071 | 3-OCH₃, 5-F | 4-CH₃ | H | H | resin |
| 1.072 | 3-F, 5-Cl | 4-CH₃ | H | H | |
| 1.073 | 3-OCH₃, 5-CF₃ | 4-CH₃ | H | H | |
| 1.074 | 3-OCH₃, 5-CN | 4-CH₃ | H | H | |
| 1.075 | 3-OCH₃, | 4-CH₃ | H | H | |
| | 5-CH═NOCH₃ | | | | |
| 1.076 | 3-OCH₃, 5-F | 4-Cl | H | H | |
| 1.077 | 3-OCH₃, 5-CF₃ | 4-Cl | H | H | |
| 1.078 | 3-OCH₃, 5-CN | 4-Cl | H | H | |
| 1.079 | 3-OCH₃, | 4-Cl | H | H | |
| | 5-CH═NOCH₃ | | | | |
| 1.080 | 3-OCH₃, 5-CF₃ | 4-CH₂CN | H | H | |
| 1.081 | 3-OCH₃, 5-CH₂F | 4-CH₂CN | H | H | |
| 1.082 | 3-OCH₃, 5-CHF₂ | 4-CH₂CN | H | H | |
| 1.083 | 3-OCH₃, 5-CF₃ | 4-OCH₂CN | H | H | |
| 1.084 | 3-OCH₃, | 4-OCH₂CN | H | H | |
| | 5-CH═NOCH₃ | | | | |
| 1.085 | 3-F, 5-Cl | 4-OCH₂CN | H | H | |
| 1.086 | 3-OCH₃, 5-F | 4-OCH₂CN | H | H | |
| 1.087 | 3-OCH₃, 5-Cl | 4-OCH₂ON | H | H | |
| 1.088 | 3-OCH₃, 5-Br | 4-OCH₂CN | H | H | |
| 1.089 | 3-OCH₃, 5-CN | 4-OCH₂CN | H | H | |
| 1.090 | 3-F, 5-F | 6-OCH₂CN | H | H | |
| 1.091 | 3-OCH₃, 5-F | 6-OCH₂CN | H | H | |
| 1.092 | 3-OCH₃, 5-CF₃ | 6-OCH₂CN | H | H | |
| 1.093 | 3-OCH₃, 5-Br | 6-OCH₂CN | H | H | |
| 1.094 | 3-OCH₃, 5-CN | 6-OCH₂CN | H | H | |
| 1.095 | 3-F, 5-F | 6-OCH₂CN | H | H | |
| 1.096 | 3-F, 5-Cl | 6-CH₂CN | H | H | |
| 1.097 | 3-OCH₃, 5-CH₂F | 6-CH₂CN | H | H | |
| 1.098 | 3-OCH₃, 5-CHF₂ | 6-CH₂CN | H | H | |
| 1.099 | 3-OCH₃, 5-CN | 6-CH₂CN | H | H | |
| 1.100 | 3-Cl, 5-CF₃ | 6-CH₂CN | H | H | |
| 1.101 | 3-OCH₃, | 6-CH₂CN | H | H | |
| | 5-CH═NOCH₃ | | | | |
| 1.102 | 3-OCH₃, 5-CF₃ | 6-CH₃ | H | H | |
| 1.103 | 3-F, 5-F | 6-CH₃ | H | H | |
| 1.104 | 3-OCH₃, 5-F | 6-CH₃ | H | H | |
| 1.105 | 3-OCH₃, 5-Cl | 6-CH₃ | H | H | |
| 1.106 | 3-OCH₃, 5-Br | 6-CH₃ | H | H | |
| 1.107 | 3-OCH₃, 5-CN | 6-CH₃ | H | H | |
| 1.108 | 3-OCH₃, | 6-CH₃ | H | H | |
| | 5-CH═NOCH₃ | | | | |
| 1.109 | 3-OCH₃, 5-CF₃ | 4-CH₃, 5-F | H | H | |
| 1.110 | 3-F, 5-F | 4-CH₃, 5-F | H | H | |
| 1.111 | 3-OCH₃, 5-F | 4-CH₃, 5-F | H | H | |
| 1.112 | 3-OCH₃, 5-Cl | 4-CH₃, 5-F | H | H | |
| 1.113 | 3-OCH₃, 5-Br | 4-CH₃, 5-F | H | H | |
| 1.114 | 3-OCH₃, 5-CN | 4-CH₃, 5-F | H | H | |
| 1.115 | 3-OCH₃, | 4-CH₃, 5-F | H | H | |
| | 5-CH═NOCH₃ | | | | |
| 1.116 | 3-OCH₃, 5-CF₃ | 4-CH₂CN | CN | H | |
| 1.117 | 3-F, 5-F | 4-CH₂CN | CN | H | |
| 1.118 | 3-OCH₃, 5-F | 4-CH₂CN | CN | H | |
| 1.119 | 3-OCH₃, 5-Cl | 4-CH₂CN | CN | H | |
| 1.120 | 3-OCH₃, 5-Br | 4-CH₂CN | CN | H | |
| 1.121 | 3-OCH₃, 5-CN | 4-CH₂CN | CN | H | |
| 1.122 | 3-OCH₃, | 4-CH₂CN | CN | H | |
| | 5-CH═NOCH₃ | | | | |
| 1.123 | 3-OCH₃, 5-CF₃ | 4-CH₂C(S)NH₂ | H | H | |
| 1.124 | 3-F, 5-F | 4-CH₂C(S)NH₂ | H | H | |
| 1.125 | 3-OCH₃, 5-F | 4-CH₂C(S)NH₂ | H | H | |
| 1.126 | 3-OCH₃, 5-Cl | 4-CH₂C(S)NH₂ | H | H | |
| 1.127 | 3-OCH₃, 5-Br | 4-CH₂C(S)NH₂ | H | H | |
| 1.128 | 3-OCH₃, 5-CN | 4-CH₂C(S)NH₂ | H | H | |
| 1.129 | 3-OCH₃, | 4-CH₂C(S)NH₂ | H | H | |
| | 5-CH═NOCH₃ | | | | |

TABLE 2
Compounds of formula I₂

| | |
|---|---|
| | (I₂) |
| 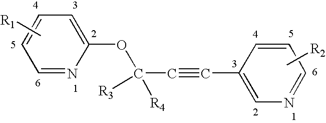 | |

| Comp. | | | | | Phys. data |
|---|---|---|---|---|---|
| No. | R₁ | R₂ | R₃ | R₄ | m.p.(° C.) |
| 2.001 | 3-F | 5-CH₂CN | H | H | |
| 2.002 | 3-Cl | 5-CH₂CN | H | H | |
| 2.003 | 3-F, 5-F | 6-Cl | H | H | |
| 2.004 | 3-F, 5-Cl | 6-Cl | H | H | |
| 2.005 | 3-OCH₃, 5-F | 6-Cl | H | H | 101-102 |
| 2.006 | 3-OCH₃, 5-Cl | 6-Cl | H | H | 100-101 |
| 2.007 | 3-OCH₃, 5-CN | 6-Cl | H | H | |
| 2.008 | 3-OCH₃, 5-CF₃ | 6-Cl | H | H | |
| 2.009 | 3-OCH₃, | 6-Cl | H | H | |
| | 5-CH═NOCH₃ | | | | |
| 2.010 | 3-F, 5-F | 5-CH(CH₃)CN | H | H | |
| 2.011 | 3-OCH₃, 5-F | 5-CH(CH₃)CN | H | H | |
| 2.012 | 3-OCH₃, 5-Cl | 5-CH(CH₃)CN | H | H | |
| 2.013 | 3-F, 5-F | 5-CH₂CN | H | H | |
| 2.014 | 3-OCH₃, 5-F | 5-CH₂CN | H | H | |
| 2.015 | 3-OCH₃, 5-Cl | 5-CH₂CN | H | H | |
| 2.016 | 3-OCH₃, | 5-CH₂CN | H | H | |
| | 5-CH═NOCH₃ | | | | |
| 2.017 | 3-F, 5-F | 6-Br | H | H | |
| 2.018 | 3-F, 5-Cl | 6-Br | H | H | |
| 2.019 | 3-OCH₃, 5-F | 6-Br | H | H | |
| 2.020 | 3-OCH₃, 5-Cl | 6-Br | H | H | |
| 2.021 | 3-OCH₃, 5-CN | 6-Br | H | H | |
| 2.022 | 3-OCH₃, 5-CF₃ | 6-Br | H | H | |
| 2.023 | 3-OCH₃, | 6-Br | H | H | |
| | 5-CH═NOCH₃ | | | | |
| 2.024 | 3-OCH₃, 5-F | 6-Cl | CH₃ | H | |
| 2.025 | 3-OCH₃, 5-F | 6-Cl | CH₃ | CH₃ | |
| 2.026 | 3-OCH₃, 5-F | 6-Cl | CN | H | |
| 2.027 | 3-OCH₃, 5-F | 6-Cl | OCH₃ | H | |
| 2.028 | 3-F, 5-F | 5-NHCOCH₃ | H | H | |
| 2.029 | 3-F, 5-Cl | 5-NHCOCH₃ | H | H | |
| 2.030 | 3-OCH₃, 5-Cl | 5-NHCOCH₃ | H | H | |
| 2.031 | 3-F, 5-Cl | 5-CHF₂ | H | H | |
| 2.032 | 3-OCH₃, 5-F | 5-CHF₂ | H | H | |
| 2.033 | 3-OCH₃, 5-Cl | 5-CHF₂ | H | H | |
| 2.034 | 3-F, 5-F | 5-C(CH₃)₂CN | H | H | |
| 2.035 | 3-OCH₃, 5-F | 5-C(CH₃)₂CN | H | H | |
| 2.036 | 3-OCH₃, 5-Cl | 5-C(CH₃)₂CN | H | H | |
| 2.037 | 3-F, 5-F | 5-CH₂CN | F | F | |
| 2.038 | 3-OCH₃, 5-F | 5-CH₂CN | F | F | |
| 2.039 | 3-OCH₃, 5-F | 5-CF₃ | H | H | |
| 2.040 | 3-OCH₃, 5-Cl | 5-CF₃ | H | H | |
| 2.041 | 3-OCH₃, 5-F | 5-NO₂ | H | H | |
| 2.042 | 3-OCH₃, 5-Cl | 5-NO₂ | H | H | |
| 2.043 | 3-OCH₃, 5-CF₃ | 5-NO₂ | H | H | |
| 2.044 | 5-CF₃ | 5-CH₂CN | H | H | |
| 2.045 | 5-CF₃ | 5-CH(CH₃)CN | H | H | |
| 2.046 | 3-OCH₃, 5-Cl | 6-OCH₃ | H | H | crystalline |
| 2.047 | 3-OCH₃, 5-Cl | H (m = 0) | H | H | solid |
| 2.048 | 3-OCH₃, 5-F | 2-Cl | H | H | 109-110 |
| 2.049 | 3-OCH₃, 5-F | 6-OCH₃ | H | H | |
| 2.050 | 3-OCH₃, 5-CF₃ | 6-OCH₃ | H | H | |
| 2.051 | 3-F, 5-F | 2-Cl | H | H | |
| 2.052 | 3-F, 5-Cl | 2-Cl | H | H | |
| 2.053 | 3-OCH₃, 5-Cl | 2-Cl | H | H | 109-110 |
| 2.054 | 3-OCH₃, 5-CN | 2-Cl | H | H | |
| 2.055 | 3-OCH₃, 5-CF₃ | 2-Cl | H | H | |
| 2.056 | 3-F, 5-F | 6-CH₃ | H | H | |
| 2.057 | 3-F, 5-Cl | 6-CH₃ | H | H | |
| 2.058 | 3-OCH₃, 5-F | 6-CH₃ | H | H | |
| 2.059 | 3-OCH₃, 5-Cl | 6-CH₃ | H | H | |
| 2.060 | 3-OCH₃, 5-CN | 6-CH₃ | H | H | |
| 2.061 | 3-OCH₃, 5-CF₃ | 6-CH₃ | H | H | |
| 2.062 | 3-OCH₃, | 6-CH₃ | H | H | |
| | 5-CH═NOCH₃ | | | | |
| 2.063 | 3-OCH₃, 5-CH₂F | 6-Cl | H | H | |
| 2.064 | 3-OCH₃, 5-CHF₂ | 6-Cl | H | H | |
| 2.065 | 3-Cl, 5-CF₃ | 6-Cl | H | H | |
| 2.066 | 3-Cl, 5-CCl₃ | 6-Cl | H | H | |
| 2.067 | 3-F, 5-F | 6-F | H | H | |
| 2.068 | 3-F, 5-Cl | 6-F | H | H | |
| 2.069 | 3-OCH₃, 5-F | 6-F | H | H | |
| 2.070 | 3-OCH₃, 5-Cl | 6-F | H | H | |
| 2.071 | 3-OCH₃, 5-CN | 6-F | H | H | |
| 2.072 | 3-OCH₃, 5-CF₃ | 6-F | H | H | |
| 2.073 | 3-OCH₃, | 6-F | H | H | |
| | 5-CH═NOCH₃ | | | | |
| 2.074 | 3-F, 5-F | 5-CH₃ | H | H | |
| 2.075 | 3-F, 5-Cl | 5-CH₃ | H | H | |
| 2.076 | 3-OCH₃, 5-F | 5-CH₃ | H | H | |
| 2.077 | 3-OCH₃, 5-Cl | 5-CH₃ | H | H | |
| 2.078 | 3-OCH₃, 5-CN | 5-CH₃ | H | H | |
| 2.079 | 3-OCH₃, 5-CF₃ | 5-CH₃ | H | H | |
| 2.080 | 3-OCH₃, | 5-CH₃ | H | H | |
| | 5-CH═NOCH₃ | | | | |
| 2.081 | 3-OCH₃, 5-F | 5-CHO | H | H | |
| 2.082 | 3-OCH₃, 5-Cl | 5-CHO | H | H | |
| 2.083 | 3-OCH₃, 5-CF₃ | 5-CHO | H | H | |
| 2.084 | 3-F, 5-F | 5-CHO | H | H | |
| 2.085 | 3-F, 5-F | 6-CHF₂ | H | H | |
| 2.086 | 3-F, 5-Cl | 6-CHF₂ | H | H | |
| 2.087 | 3-OCH₃, 5-F | 6-CHF₂ | H | H | |
| 2.088 | 3-OCH₃, 5-Cl | 6-CHF₂ | H | H | |
| 2.089 | 3-OCH₃, 5-CN | 6-CHF₂ | H | H | |
| 2.090 | 3-OCH₃, 5-CF₃ | 6-CHF₂ | H | H | |
| 2.091 | 3-OCH₃, | 6-CHF₂ | H | H | |
| | 5-CH═NOCH₃ | | | | |
| 2.092 | 3-F, 5-F | 6-OCH₂CN | H | H | |
| 2.093 | 3-F, 5-Cl | 6-OCH₂CN | H | H | |
| 2.094 | 3-OCH₃, 5-F | 6-OCH₂CN | H | H | |
| 2.095 | 3-OCH₃, 5-Cl | 6-OCH₂CN | H | H | |
| 2.096 | 3-OCH₃, 5-CN | 6-OCH₂CN | H | H | |
| 2.097 | 3-OCH₃, 5-CF₃ | 6-OCH₂CN | H | H | |
| 2.098 | 3-OCH₃, | 6-OCH₂CN | H | H | |
| | 5-CH═NOCH₃ | | | | |
| 2.099 | 3-F, 5-F | 6-CH₂CN | H | H | |
| 2.100 | 3-F, 5-Cl | 6-CH₂CN | H | H | |
| 2.101 | 3-OCH₃, 5-F | 6-CH₂CN | H | H | |
| 2.102 | 3-OCH₃, 5-Cl | 6-CH₂CN | H | H | |
| 2.103 | 3-OCH₃, 5-CN | 6-CH₂CN | H | H | |
| 2.104 | 3-OCH₃, 5-CF₃ | 6-CH₂CN | H | H | |
| 2.105 | 3-OCH₃, | 6-CH₂CN | H | H | |
| | 5-CH═NOCH₃ | | | | |
| 2.106 | 3-F, 5-F | 6-Cl | CN | H | |
| 2.107 | 3-F, 5-Cl | 6-Cl | CN | H | |
| 2.108 | 3-Cl, 5-CF₃ | 6-Cl | CN | H | |
| 2.109 | 3-OCH₃, 5-Cl | 6-Cl | CN | H | |
| 2.110 | 3-OCH₃, 5-CN | 6-Cl | CN | H | |
| 2.111 | 3-OCH₃, 5-CF₃ | 6-Cl | CN | H | |
| 2.112 | 3-OCH₃, | 6-Cl | CN | H | |
| | 5-CH═NOCH₃ | | | | |
| 2.113 | 3-F, 5-F | 5-CH₂C(S)NH₂ | H | H | |
| 2.114 | 3-F, 5-Cl | 5-CH₂C(S)NH₂ | H | H | |
| 2.115 | 3-Cl, 5-CF₃ | 5-CH₂C(S)NH₂ | H | H | |
| 2.116 | 3-OCH₃, 5-Cl | 5-CH₂C(S)NH₂ | H | H | |
| 2.117 | 3-OCH₃, 5-CN | 5-CH₂C(S)NH₂ | H | H | |
| 2.118 | 3-OCH₃, 5-CF₃ | 5-CH₂C(S)NH₂ | H | H | |
| 2.119 | 3-OCH₃, | 5-CH₂C(S)NH₂ | H | H | |
| | 5-CH═NOCH₃ | | | | |

TABLE 3
Compounds of formula I₃

| | |
|---|---|
| | (I₃) |
| 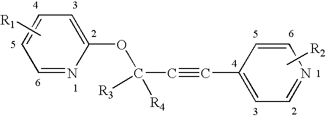 | |

| Comp. | | | | | Phys. data |
|---|---|---|---|---|---|
| No. | R₁ | R₂ | R₃ | R₄ | m.p.(° C.) |
| 3.001 | 3-F | 2-Cl | H | H | |
| 3.002 | 3-Cl | 2-Cl | H | H | |
| 3.003 | 3-F, 5-F | 2-Cl | H | H | |
| 3.004 | 3-F, 5-Cl | 2-Cl | H | H | |
| 3.005 | 3-OCH₃, 5-F | 2-Cl | H | H | |
| 3.006 | 3-OCH₃, 5-Cl | 2-Cl | H | H | |
| 3.007 | 3-OCH₃, 5-CN | 2-Cl | H | H | |
| 3.008 | 3-OCH₃, 5-CF₃ | 2-Cl | H | H | |
| 3.009 | 3-OCH₃, | 2-Cl | H | H | |
| | 5-CH═NOCH₃ | | | | |
| 3.010 | 3-F, 5-F | 2-CH₃ | H | H | |
| 3.011 | 3-F, 5-Cl | 2-CH₃ | H | H | |
| 3.012 | 3-OCH₃, 5-F | 2-CH₃ | H | H | |
| 3.013 | 3-OCH₃, 5-Cl | 2-CH₃ | H | H | |
| 3.014 | 3-OCH₃, 5-CN | 2-CH₃ | H | H | |
| 3.015 | 3-OCH₃, 5-CF₃ | 2-CH₃ | H | H | |
| 3.016 | 3-OCH₃, 5-CF₃ | 2-CH(CH₃)CN | H | H | |
| 3.017 | 3-OCH₃, | 2-CH(CH₃)CN | H | H | |
| | 5-CH═NOCH₃ | | | | |
| 3.018 | 3-F, 5-F | 2-CH(CH₃)CN | H | H | |
| 3.019 | 3-F, 5-Cl | 2-CH(CH₃)CN | H | H | |
| 3.020 | 3-OCH₃, 5-F | 2-CH(CH₃)CN | H | H | |
| 3.021 | 3-OCH₃, 5-Cl | 2-CH(CH₃)CN | H | H | |
| 3.022 | 3-OCH₃, 5-CN | 2-CH(CH₃)CN | H | H | |
| 3.023 | 3-OCH₃, 5-F | 3-Cl, 6-Cl | CH₃ | H | |
| 3.024 | 3-OCH₃, 5-Cl | 3-Cl, 6-CH₃ | CH₃ | H | |
| 3.025 | 3-OCH₃, 5-CN | 3-Cl, 6-CHF₂ | CH₃ | H | |
| 3.026 | 3-OCH₃, 5-CF₃ | 3-Cl, 6-OCH₂CN | CH₃ | H | |
| 3.027 | 3-OCH₃, | 3-Cl, 6-CH₂CN | CH₃ | H | |
| | 5-CH═NOCH₃ | | | | |
| 3.028 | 3-F, 5-F | 2-Cl, 6-F | H | H | |
| 3.029 | 3-F, 5-Cl | 2-Cl, 6-F | H | H | |
| 3.030 | 3-OCH₃, 5-F | 2-Cl, 6-F | H | H | |
| 3.031 | 3-OCH₃, 5-Cl | 2-Cl, 6-F | H | H | |
| 3.032 | 3-OCH₃, 5-CN | 2-Cl, 6-F | H | H | |
| 3.033 | 3-F, 5-F | 2-OH(CH₃)CN | F | F | |
| 3.034 | 3-F, 5-Cl | 2-CH(CH₃)CN | F | F | |
| 3.035 | 3-OCH₃, 5-F | 2-CH(CH₃)CN | F | F | |
| 3.036 | 3-OCH₃, 5-Cl | 2-CH(CH₃)CN | F | F | |
| 3.037 | 3-OCH₃, 5-CN | 2-CH(CH₃)CN | F | F | |
| 3.038 | 3-F | 2-CH₂CN | H | H | |
| 3.039 | 3-Cl | 2-CH₂CN | H | H | |
| 3.040 | 3-F, 5-F | 2-CH₂CN | H | H | |
| 3.041 | 3-F, 5-Cl | 2-CH₂CN | H | H | |
| 3.042 | 3-OCH₃, 5-F | 2-CH₂CN | H | H | |
| 3.043 | 3-OCH₃, 5-Cl | 2-CH₂CN | H | H | |
| 3.044 | 3-OCH₃, 5-CN | 2-CH₂CN | H | H | |
| 3.045 | 3-OCH₃, 5-CF₃ | 2-CH₂CN | H | H | |
| 3.046 | 3-OCH₃, | 2-CH₂CN | H | H | |
| | 5-CH═NOCH₃ | | | | |
| 3.047 | 3-OCH₃, 5-F | 2-OCH₂CN | H | H | |
| 3.048 | 3-OCH₃, 5-Cl | 2-OCH₂CN | H | H | |
| 3.049 | 3-OCH₃, 5-CN | 2-OCH₂CN | H | H | |
| 3.050 | 3-OCH₃, 5-CF₃ | 2-OCH₂CN | H | H | |
| 3.051 | 3-OCH₃, | 2-OCH₂CN | H | H | |
| | 5-CH═NOCH₃ | | | | |
| 3.052 | 3-F, 5-F | 2-CHF₂ | H | H | |
| 3.053 | 3-F, 5-Cl | 2-CHF₂ | H | H | |
| 3.054 | 3-OCH₃, 5-F | 2-CHF₂ | H | H | |
| 3.055 | 3-OCH₃, 5-Cl | 2-CHF₂ | H | H | |
| 3.056 | 3-OCH₃, 5-CN | 2-CHF₂ | H | H | |
| 3.057 | 3-OCH₃, 5-CF₃ | 2-CHF₂ | H | H | |
| 3.058 | 3-F, 5-F | 2-CHO | H | H | |
| 3.059 | 3-F, 5-Cl | 2-CHO | H | H | |
| 3.060 | 3-OCH₃, 5-F | 2-CHO | H | H | |
| 3.061 | 3-OCH₃, 5-Cl | 2-CHO | H | H | |
| 3.062 | 3-OCH₃, 5-CN | 2-CHO | H | H | |
| 3.063 | 3-OCH₃, 5-CF₃ | 2-CHO | H | H | |
| 3.064 | 3-OCH₃, 5-F | 2-CF₃ | H | H | |
| 3.065 | 3-OCH₃, 5-Cl | 2-CF₃ | H | H | |
| 3.066 | 3-OCH₃, 5-CN | 2-CF₃ | H | H | |
| 3.067 | 3-OCH₃, 5-CF₃ | 2-CF₃ | H | H | |
| 3.068 | 3-OCH₃, | 2-CF₃ | H | H | |
| | 5-CH═NOCH₃ | | | | |
| 3.069 | 3-F | 3-Cl | H | H | |
| 3.070 | 3-Cl | 3-Cl | H | H | |
| 3.071 | 3-F, 5-F | 3-Cl | H | H | |
| 3.072 | 3-F, 5-Cl | 3-Cl | H | H | |
| 3.073 | 3-OCH₃, 5-F | 3-Cl | H | H | |
| 3.074 | 3-OCH₃, 5-Cl | 3-Cl | H | H | |
| 3.075 | 3-OCH₃, 5-CN | 3-Cl | H | H | |
| 3.076 | 3-OCH₃, 5-CF₃ | 3-Cl | H | H | |
| 3.077 | 3-OCH₃, | 3-Cl | H | H | |
| | 5-CH═NOCH₃ | | | | |
| 3.078 | 3-OCH₃, 5-F | 2-CO₂C₂H₅ | H | H | |
| 3.079 | 3-OCH₃, 5-Cl | 2-CO₂C₂H₅ | H | H | |
| 3.080 | 3-OCH₃, 5-CN | 2-CO₂C₂H₅ | H | H | |
| 3.081 | 3-OCH₃, 5-CF₃ | 2-CO₂C₂H₅ | H | H | |
| 3.082 | 3-OCH₃, | 2-CO₂C₂H₅ | H | H | |
| | 5-CH═NOCH₃ | | | | |
| 3.083 | 3-F | 2-CH₂C(S)NH₂ | H | H | |
| 3.084 | 3-Cl | 2-CH₂C(S)NH₂ | H | H | |
| 3.085 | 3-F, 5-F | 2-CH₂C(S)NH₂ | H | H | |
| 3.086 | 3-F, 5-Cl | 2-CH₂C(S)NH₂ | H | H | |
| 3.087 | 3-OCH₃, 5-F | 2-CH₂C(S)NH₂ | H | H | |
| 3.088 | 3-OCH₃, 5-Cl | 2-CH₂C(S)NH₂ | H | H | |
| 3.089 | 3-OCH₃, 5-CN | 2-CH₂C(S)NH₂ | H | H | |
| 3.090 | 3-OCH₃, 5-CF₃ | 2-CH₂C(S)NH₂ | H | H | |
| 3.091 | 3-OCH₃, | 2-CH₂C(S)NH₂ | H | H | |
| | 5-CH═NOCH₃ | | | | |
| 3.092 | 3-F, 5-Cl | 2-CH₂CO₂H | H | H | |
| 3.093 | 3-OCH₃, 5-F | 2-CH₂CO₂H | H | H | |
| 3.094 | 3-OCH₃, 5-Cl | 2-CH₂CO₂H | H | H | |
| 3.095 | 3-OCH₃, 5-CN | 2-CH₂CO₂H | H | H | |
| 3.096 | 3-OCH₃, 5-CF₃ | 2-CH₂CO₂H | H | H | |
| 3.097 | 3-OCH₃, | 2-CH₂CO₂H | H | H | |
| | 5-CH═NOCH₃ | | | | |

### Biological Examples

### Example B1

### Herbicidal Action Prior to Emergence of the Plants (Pre-Emergence Action)

Monocotyledonous and dicotyledonous test plants are sown in standard soil in pots. Immediately after sowing, the test compounds, in the form of an aqueous suspension (prepared from a wettable powder (Example F3, b) according to WO 97/34485) or in the form of an emulsion (prepared from an emulsifiable concentrate (Example F1, c) according to WO 97/34485), are applied by spraying in an optimum concentration (500 litres of water/ha). The test plants are then grown in a greenhouse under optimum conditions. After a test duration of 4 weeks, the test is evaluated in accordance with a scale of nine ratings (1=total damage, 9=no action). Ratings of from 1 to 4 (especially from 1 to 3) indicate good to very good herbicidal action.

Test plants: Setaria (Seta), Panicum (Pani), Echinochloa (Ds) (Echino), Amaranthus (Amar), Chenopodium (Cheno), Stellaria (Stella), Veronica, Euphorbia (Eupho), Brachiaria (Brachi).

TABLE B1
Concentration 1000 g of active ingredient/ha

| Comp. | | | Echino | | | | | | |
|---|---|---|---|---|---|---|---|---|---|
| No. | Seta | Pani | (Ds) | Amar | Cheno | Stella | Veronica | Eupho | Brachi |
| 1.063 | 2 | 1 | 4 | 1 | — | — | — | 1 | 1 |
| 2.005 | 4 | 4 | 1 | — | 1 | 1 | 4 | 5 | 1 |
| 2.006 | 1 | 1 | 1 | 1 | — | 1 | 5 | 1 | 1 |

The same results are obtained when the compounds of formula I are formulated in accordance with the other Examples analogously to WO 97/34485.

### Example B2

### Post-Emergence Herbicidal Action

Monocotyledonous and dicotyledonous test plants are sown in standard soil in pots. When the test plants are at the 2- to 3-leaf stage, the test compounds, in the form of an aqueous suspension (prepared from a wettable powder (Example F3, b) according to WO 97/34485) or in the form of an emulsion (prepared from an emulsifiable concentrate (Example F1, c) according to WO 97/34485), are applied by spraying in an optimum concentration (500 litres of water/ha). The test plants are then grown on in a greenhouse under optimum conditions. After a test duration of 2 to 3 weeks, the test is evaluated in accordance with a scale of nine ratings (1=total damage, 9=no action). Ratings of from 1 to 4 (especially from 1 to 3) indicate good to very good herbicidal action.

Test plants: Setaria (Seta), Panicum (Pani), Echinochloa (Ds) (Echino), Amaranthus (Amar), Chenopodium (Cheno), Stellaria (Stella), Veronica, Euphorbia (Eupho).

TABLE B2
Concentration 1000 g of active ingredient/ha

| Comp. | | | Echino | | | | | |
|---|---|---|---|---|---|---|---|---|
| No. | Seta | Pani | (Ds) | Amar | Cheno | Stella | Veronica | Eupho |
| 1.063 | 5 | 3 | 3 | 2 | 2 | — | — | 1 |
| 2.005 | 3 | 4 | — | 1 | 3 | 2 | 2 | 1 |
| 2.006 | 2 | 2 | — | 2 | 2 | 2 | 2 | 1 |

In the above Tables B1 and B2 “—” means that no data are available for that indication.

The same results are obtained when the compounds of formula I are formulated in accordance with the other Examples analogously to WO 97/34485.

1. A compound of formula I 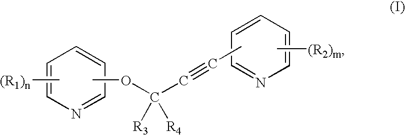
wherein
n is 0, 1, 2, 3 or 4;
each R₁ independently of any others is halogen, —CN, —SCN, —SF₅, —NO₂, —NR₅R₆, —CO₂R₇, —CONR₈R₉, —C(R₁₀)═NOR₁₁, —COR₁₂, —OR₁₃, —SR₁₄, —SOR₁₅, —SO₂R₆, —SO₂R₁₇, C₁-C₈alkyl, C₂-C₈alkenyl, C₂-C₈alkynyl or C₃-C₆cycloalkyl; or is C₁-C₈alkyl, C₂-C₈alkenyl or C₂-C₈alkynyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)=NR₂₈, —OR₂₉, —SR₃₀, —SOR₃₁, —SO₂R₃₂ or C₃-C₆cycloalkyl substituents; or
each R₁ independently of any others is C₃-C₆cycloalkyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)=NR₂₈, —SR₃₀, —SOR₃₁, —SO₂R₃₂ or C₃-C₆cycloalkyl substituents; or
each R₁ independently of any others is phenyl, which may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
two adjacent R₁ together form a C₁-C₇alkylene bridge, which may be interrupted by 1 or 2 non-adjacent oxygen atoms and may be substituted by C₁-C₆alkyl or C₁-C₆alkoxy, the total number of ring atoms being at least 5 and at most 9; or
two adjacent R₁ together form a C₂-C₇alkenylene bridge, which may be interrupted by 1 or 2 non-adjacent oxygen atoms and may be substituted by C₁-C₆alkyl or C₁-C₆alkoxy, the total number of ring atoms being at least 5 and at most 9;
R₃ and R₄ are each independently of the other hydrogen, halogen, —CN, C₁-C₄alkyl or C₁-C₄alkoxy; or
R₃ and R₄ together are C₂-C₅alkylene;
R₅ is hydrogen, C₁-C₈alkyl or —C(O)C₁-C₈alkyl;
R₆ is hydrogen, C₁-C₈alkyl, C₃-C₈alkenyl, C₃-C₈alkynyl, phenyl or benzyl; wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₅ and R₆ together are a C₂-C₅alkylene chain, which may be interrupted by an oxygen or a sulfur atom;
R₇ is hydrogen, C₁-C₈alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₈ is hydrogen or C₁-C₈alkyl;
R₉ is hydrogen or C₁-C₈alkyl, or is C₁-C₈alkyl substituted by one or more —COOH, C₁-C₈alkoxycarbonyl or —CN substituents, or
R₉ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₈ and R₉ together are C₂-C₅alkylene;
R₁₀ is hydrogen, C₁-C₄alkyl, C₁-C₄haloalkyl or C₃-C₆cycloalkyl;
R₁₁ is hydrogen, C₁-C₈alkyl, C₃-C₈alkenyl, C₃-C₈alkynyl, C₁-C₄haloalkyl or C₃-C₆haloalkenyl;
R₁₂ is hydrogen, C₁-C₄alkyl, C₁-C₄haloalkyl or C₃-C₆cycloalkyl;
R₁₃ is hydrogen, C₁-C₈alkyl, C₃-C₈alkenyl or C₃-C₈alkynyl; or
R₁₃ is phenyl or phenyl-C₁-C₆alkyl, wherein the phenyl ring may in turn be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents, or
R₁₃ is C₁-C₈alkyl substituted by one or more halogen, —CN, C₁-C₆alkylamino, di(C₁-C₆alkyl)amino or C₁-C₄alkoxy substituents;
R₁₄ is hydrogen, C₁-C₈alkyl, C₃-C₈alkenyl or C₃-C₈alkynyl, or is C₁-C₈alkyl substituted by one or more halogen, —CN or C₁-C₄alkoxy substituents;
R₁₅, R₁₆ and R₁₇ are each independently of the others C₁-C₈alkyl, C₃-C₈alkenyl or C₃-C₈alkynyl, or C₁-C₈alkyl substituted by one or more halogen, —CN or C₁-C₄alkoxy substituents;
R₁₈ is hydrogen or C₁-C₈alkyl;
R₁₉ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₈ and R₁₉ together are a C₂-C₅alkylene chain, which may be interrupted by an oxygen or a sulfur atom;
R₂₀ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₂₁ is hydrogen or C₁-C₈alkyl;
R₂₂ is hydrogen or C₁-C₈alkyl, or is C₁-C₈alkyl substituted by one or more —COOH, C₁-C₈-alkoxycarbonyl or —CN substituents, or
R₂₂ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₂₁ and R₂₂ together are C₂-C₅alkylene;
R₂₃ is hydrogen, C₁-C₄alkyl, C₁-C₄haloalkyl or C₃-C₆cycloalkyl;
R₂₄ is hydrogen, C₁-C₄alkyl, C₁-C₄haloalkyl or C₃-C₆cycloalkyl;
R₂₅ is hydrogen, C₁-C₈alkyl, C₃-C₈alkenyl, C₃-C₈alkynyl, C₁-C₄haloalkyl or C₃-C₆haloalkenyl;
R₂₆ is hydrogen or C₁-C₈alkyl;
R₂₇ is hydrogen or C₁-C₈alkyl, or is C₁-C₈alkyl substituted by one or more —COOH, C₁-C₈-alkoxycarbonyl or —CN substituents, or
R₂₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₂₆ and R₂₇ together are C₂-C₅alkylene;
R₂₈ is hydrogen or C₁-C₈alkyl;
R₂₉ and R₃₀ are each independently of the other hydrogen, C₁-C₈alkyl, C₃-C₈alkenyl or C₃-C₈alkynyl, or C₁-C₈alkyl substituted by one or more halogen, —CN or C₁-C₄alkoxy substituents;
R₃₁ and R₃₂ are each independently of the other C₁-C₈alkyl, C₃-C₈alkenyl or C₃-C₈alkynyl, or C₁-C₈alkyl substituted by one or more halogen, —CN or C₁-C₄alkoxy substituents;
m is 0, 1, 2, 3 or 4;
each R₂ independently of any others is halogen, —CN, —SCN, —OCN, —N₃, —SF₅, —NO₂, —NR₃₃R₃₄, —CO₂R₃₅, —CONR₃₆R₃₇, —C(R₃₈)═NOR₃₉, —COR₄₀, —OR₄₁, —SR₄₂, —SOR₄₃, —SO₂R₄₄, OSO₂R₄₅, —N([CO]ₚR₄₆)COR₄₇, —N(OR₅₄)COR₅₅, —N(R₅₆)SO₂R₅₇, —N(SO₂R₅₈)SO₂R₅₉, —N═C(OR₆₀)R₆₁, —CR₆₂(OR₆₃)OR₆₄, —OC(O)NR₆₅R₆₆, —SC(O)NR₆₇R₆₈, —OC(S)NR₆₉R₇₀ or —N-phthalimide; or
R₂ is a 5- to 7-membered heterocyclic ring system, which may be aromatic or partially or fully saturated and may contain from 1 to 4 hetero atoms selected from nitrogen, oxygen and sulfur, it being possible for that heterocyclic ring system in turn to be substituted by one or more halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or C₁-C₆alkylsulfonyl substituents;
R₃₃ is hydrogen or C₁-C₈alkyl; and
R₃₄ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₃₃ and R₃₄ together are a C₂-C₅alkylene chain, which may be interrupted by an oxygen or a sulfur atom;
R₃₅ is hydrogen, C₁-C₈alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₃₆ is hydrogen or C₁-C₈alkyl;
R₃₇ is hydrogen or C₁-C₈alkyl, or is C₁-C₈alkyl substituted by one or more —COOH, C₁-C₈-alkoxycarbonyl or —CN substituents, or
R₃₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₃₆ and R₃₇ together are C₃-C₅alkylene;
R₃₈ is hydrogen, C₁-C₄alkyl, C₁-C₄haloalkyl or C₃-C₆cycloalkyl;
R₃₉ is hydrogen, C₁-C₈alkyl, C₃-C₈alkenyl, C₃-C₈alkynyl, C₁-C₄haloalkyl or C₃-C₆haloalkenyl;
R₄₀ is hydrogen, C₁-C₄alkyl, C₁-C₄haloalkyl, C₁-C₈alkylthio, —C(O)—C(O)OC₁-C₄alkyl or C₃-C₆-cycloalkyl;
R₄₁ is hydrogen, C₁-C₈alkyl, C₃-C₈alkenyl, C₃-C₈alkynyl, C₁-C₆alkoxy-C₁-C₆alkyl, C₁-C₈alkylcarbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or
R₄₁ is phenyl or phenyl-C₁-C₆alkyl, wherein the phenyl ring may in turn be substituted by one or more halogen, C₁-C₄alkyl, C₁-C₄haloalkyl, C₁-C₄alkoxy, —CN, —NO₂ or —S(O)₂C₁-C₈alkyl substituents, or
R₄₁ is C₁-C₈alkyl substituted by one or more halogen, —COOH, C₁-C₈alkoxycarbonyl, C₁-C₆alkylamino, di(C₁-C₆alkyl)amino or —CN substituents;
R₄₂ is hydrogen, C₁-C₈alkyl, C₃-C₈alkenyl or C₃-C₈alkynyl, or is C₁-C₈alkyl substituted by one or more halogen, —CN or C₁-C₄alkoxy substituents;
R₄₃ and R₄₄ are each independently of the other C₁-C₈alkyl, C₃-C₈alkenyl or C₃-C₈alkynyl, or C₁-C₈alkyl substituted by one or more halogen, —CN or C₁-C₄alkoxy substituents;
R₄₅ is C₁-C₈alkyl, C₁-C₈alkyl substituted by one or more halogen, —CN or C₁-C₄alkoxy substituents, C₃-C₈alkenyl or C₃-C₈alkynyl, or
R₄₅ is phenyl, it being possible for the phenyl ring to be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈-alkylsulfonyl substituents;
R₄₆ is hydrogen, C₁-C₈alkyl, C₃-C₈alkenyl, C₃-C₈alkynyl or C₁-C₄haloalkyl;
R₄₇ is hydrogen, C₁-C₈alkyl, C₁-C₄alkoxy, C₃-C₈alkenyl or C₃-C₈alkynyl, or is C₁-C₈alkyl substituted by one or more halogen, —CN, C₁-C₄alkoxy, C₁-C₈alkoxycarbonyl, —NH₂, C₁-C₄alkylamino, di(C-C₄alkyl)amino, —NR₄₈COR₄₉, —NR₅₀SO₂R₅₁ or —NR₅₂CO₂R₅₃ substituents, or
R₄₇ is phenyl or benzyl, each of which may in turn be substituted by one or more halogen, C₁-C₄alkyl, C₁-C₄haloalkyl, C₁-C₄alkoxy, —CN, —NO₂, Cl-C₄alkylthio, C₁-C₄alkylsulfinyl or C₁-C₄alkylsulfonyl substituents;
p is 0 or 1;
R₄₈, R₄₉, R₅₀, R₅₁, R₅₂ and R₅₃ are each independently of the others hydrogen, C₁-C₈alkyl, phenyl, benzyl or naphthyl, it being possible for the three last-mentioned aromatic radicals in turn to be substituted by one or more halogen, C₁-C₈alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;
R₅₄ and R₅₅ are each independently of the other hydrogen, C₁-C₈alkyl or phenyl, which may in turn be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;
R₅₆ is hydrogen, C₁-C₈alkyl, C₁-C₄haloalkyl, C₁-C₄alkoxy, C₃-C₈alkenyl, C₃-C₈alkynyl or benzyl, it being possible for benzyl in turn to be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;
R₅₇ is C₁-C₈alkyl, C₁-C₄haloalkyl, phenyl, benzyl or naphthyl, it being possible for the three last-mentioned aromatic rings to be substituted by one or more halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;
R₅₈ and R₅₉ are each independently of the other C₁-C₈alkyl, C₃-C₈alkenyl, C₃-C₈alkynyl, phenyl, benzyl or naphthyl, it being possible for the three last-mentioned aromatic rings to be substituted by one or more halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;
R₆₀ and R₆, are each independently of the other hydrogen or C₁-C₆alkyl;
R₆₂, R₆₃ and R₆₄ are each independently of the others hydrogen or C₁-C₈alkyl, or R₆₃ and R₆₄ together form a C₂-C₅alkylene bridge;
R₆₅, R₆₆, R₆₇, R₆₈, R₆₉ and R₇₀ are each independently of the others hydrogen or C₁-C₈alkyl, or R₆₅ and R₆₆ together or R₆₇ and R₆₈ together or R₆₉ and R₇₀ together form a C₂-C₅alkylene bridge; or each R₂ independently of any others is C₁-C₈alkyl, or is C₁-C₈alkyl mono- or poly-substituted by halogen, —CN, —N₃, —SCN, —NO₂, —NR₇₁R₇₂, —CO₂R₇₃, —CONR₇₄R₇₅, —COR₇₆, —C(R₇₇)═NOR₇₈, —C(S)NR₇₉R₈₀, —C(C₁-C₄alkylthio)=NR₈₁, —OR₈₂, —SR₈₃, —SOR₈₄, —SO₂R₈₅, —O(SO₂R₈₆, N(R₈₇)CO₂R₈₈, —N(R₈₉)COR₉₀, —S⁺(R₉₁)₂, —N⁺(R₉₂)₃, —Si(R₉₃)₃ or C₃-C₆cycloalkyl; or
each R₂ independently of any others is C₁-C₈alkyl substituted by a 5- to 7-membered heterocyclic ring system, which may be aromatic or partially or fully saturated and may contain from 1 to 4 hetero atoms selected from nitrogen, oxygen and sulfur, it being possible for that heterocyclic ring system in turn to be substituted by one or more halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or C₁-C₆alkylsulfonyl substituents; or
each R₂ independently of any others is C₂-C₈alkenyl, or is C₂-C₈alkenyl mono- or poly-substituted by —CN, —NO₂, —CO₂R₉₄, —CONR₉₅R₉₆, —COR₉₇, —C(R₉₈)═NOR₉₉, —C(S)NR₁₀₀R₁₀₁, —C(C₁-C₄alkylthio)=NR₁₀₂, —OR₁₀₃, —Si(R₁₀₄)₃ or C₃-C₆cycloalkyl; or
each R₂ independently of any others is C₂-C₈alkynyl, or is C₂-C₈alkynyl mono- or poly-substituted by halogen, —CN, —CO₂R₁₀₅, —CONR₁₀₆R₁₀₇, —COR₁₀₈, —C(R₁₀₉)═NOR₁₁₀, —(S)NR₁₁₁R₁₁₂, —C(C₁-C₄alkylthio)=NR₁₁₃, —OR₁₁₄, —Si(R₁₁₅)₃ or C₃-C₆cycloalkyl; or
each R₂ independently of any others is C₃-C₆cycloalkyl, or is C₃-C₆cycloalkyl mono- or poly-substituted by halogen, —CN, —CO₂R₁₁₆, —CONR₁₁₇R₁₁₈, —COR₁₁₉, —C(R₁₂₀)═NOR₁₂₁, —C(S)NR₁₂₂R₁₂₃ or —C(C₁-C₄alkylthio)=NR₁₂₄; or
two adjacent R₂ together form a C₁-C₇alkylene bridge, which may be interrupted by 1 or 2 non-adjacent oxygen atoms and may be substituted by cyano, C₁-C₆alkyl or C₁-C₆alkoxy, the total number of ring atoms being at least 5 and at most 9; or
two adjacent R₂ together form a C₂-C₇alkenylene bridge, which may be interrupted by 1 or 2 non-adjacent oxygen atoms and may be substituted by cyano, C₁-C₆alkyl or C₁-C₆alkoxy, the total number of ring atoms being at least 5 and at most 9;
R₇₁ is hydrogen or C₁-C₈alkyl;
R₇₂ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₇₁ and R₇₂ together are a C₂-C₅alkylene chain, which may be interrupted by an oxygen or a sulfur atom;
R₇₃ is hydrogen, C₁-C₈alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, it being possible for phenyl in turn to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₇₄ is hydrogen or C₁-C₈alkyl;
R₇₅ is hydrogen, C₁-C₈alkyl or C₃-C₇cycloalkyl, or is C₁-C₈alkyl substituted by one or more —COOH, C₁-C₈alkoxycarbonyl, C₁-C₆alkoxy or —CN substituents; or
R₇₅ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₇₄ and R₇₅ together are a C₂-C₅alkylene chain, which may be interrupted by an oxygen or sulfur atom;
R₇₆ is hydrogen, C₁-C₄alkyl, C₁-C₄haloalkyl or C₃-C₆cycloalkyl;
R₇₇ is hydrogen, C₁-C₄alkyl, C₁-C₄haloalkyl or C₃-C₆cycloalkyl;
R₇₈ is hydrogen, C₁-C₈alkyl, C₃-C₈alkenyl, C₃-C₈alkynyl, C₁-C₄haloalkyl or C₃-C₆haloalkenyl; and
R₇₉ is hydrogen or C₁-C₈alkyl;
R₈₀ is hydrogen or C₁-C₈alkyl, or is C₁-C₈alkyl substituted by one or more —COOH, C₁-C₈-alkoxycarbonyl or —CN substituents; or
R₈₀ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₇₉ and R₈₀ together are C₂-C₅alkylene;
R₈₁ is hydrogen or C₁-C₈alkyl;
R₈₂ is —Si(C₁-C₆alkyl)₃, C₃-C₈alkenyl, C₃-C₈alkynyl or C₁-C₈alkyl, which is mono- or poly-substituted by halogen, —CN, —NH₂, C₁-C₆alkylamino, di(C₁-C₆alkyl)amino or C₁-C₄alkoxy;
R₈₃ is hydrogen, C₁-C₈alkyl, C₃-C₈alkenyl, C₃-C₈alkynyl or C₁-C₈alkyl, which is mono- or poly-substituted by halogen, —CN, —NH₂, C₁-C₆alkylamino, di(C₁-C₆alkyl)amino or C₁-C₄alkoxy;
R_{84,} R₈₅ and R₈₆ are each independently of the others C₁-C₈alkyl, C₃-C₈alkenyl or C₃-C₈alkynyl, or C₁-C₈alkyl which is substituted by one or more halogen, —CN or C₁-C₄alkoxy substituents;
R₈₇ and R₈₉ are each independently of the other hydrogen, C₁-C₈alkyl or C₁-C₈alkoxy;
R₈₈ is C₁-C₈alkyl;
R₉₀ is hydrogen or C₁-C₈alkyl;
R₉₁ is C₁-C₄alkyl;
R₉₂ and R₉₃ are each independently of the other C₁-C₆alkyl;
R₉₄ is hydrogen, C₁-C₈alkyl, C₃-C₈alkenyl or C₃-C₈alkynyl, wherein the last 3 substituents may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₉₅ is hydrogen or C₁-C₈alkyl;
R₉₆ is hydrogen or C₁-C₈alkyl, or is C₁-C₈alkyl substituted by one or more —COOH, C₁-C₈—alkoxycarbonyl or —CN substituents; or
R₉₆ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₉₅ and R₉₆ together are C₂-C₅alkylene;
R₉₇ and R₉₈ are each independently of the other hydrogen, C₁-C₄alkyl, C₁-C₄haloalkyl or C₃-C₆cycloalkyl;
R₉₉ is hydrogen, C₁-C₈alkyl, C₃-C₈alkenyl, C₃-C₈alkynyl, C₁-C₄haloalkyl or C₃-C₆haloalkenyl;
R₁₀₀ is hydrogen or C₁-C₈alkyl;
R₁₀₁ is hydrogen or C₁-C₈alkyl, or is C₁-C₈alkyl substituted by one or more —COOH, C₁-C₈-alkoxycarbonyl or —CN substituents; or
R₁₀₁ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₀₀ and R₁₀₁ together are C₂-C₅alkylene;
R₁₀₂ is hydrogen or C₁-C₈alkyl;
R₁₀₃ is hydrogen, C₁-C₈alkyl, —Si(C₁-C₆alkyl)₃, C₃-C₈alkenyl or C₃-C₈alkynyl;
R104 is C₁-C₆alkyl;
R₁₀₅ is hydrogen, C₁-C₈alkyl, C₃-C₈alkenyl or C₃-C₈alkynyl, wherein the last 3 substituents may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₁₀₆ is hydrogen or C₁-C₈alkyl;
R₁₀₇ is hydrogen or C₁-C₈alkyl, or is C₁-C₈alkyl substituted by one or more —COOH, C₁-C₈-alkoxycarbonyl or —CN substituents; or
R₁₀₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₀₆ and R₁₀₇ together are C₂-C₅alkylene;
R₁₀₈ is hydrogen, C₁-C₄alkyl, C₁-C₄haloalkyl or C₃-C₆cycloalkyl;
R₁₀₉ is hydrogen, C₁-C₄alkyl, C₁-C₄haloalkyl or C₃-C₆cycloalkyl;
R₁₁₀ is hydrogen, C₁-C₈alkyl, C₃-C₈alkenyl, C₃-C₈alkynyl, C₁-C₄haloalkyl or C₃-C₆haloalkenyl;
R₁₁₁ is hydrogen or C₁-C₈alkyl;
R₁₁₂ is hydrogen or C₁-C₈alkyl, or is C₁-C₈alkyl substituted by one or more —COOH, C₁-C₈-alkoxycarbonyl or —CN substituents; or
R₁₁₂ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₁₁ and R₁₁₂ together are C₂-C₅alkylene;
R₁₁₃ is hydrogen or C₁-C₈alkyl;
R₁₁₄ is hydrogen, C₁-C₈alkyl, —Si(C₁-C₆alkyl)₃, C₃-C₈alkenyl or C₃-C₈alkynyl;
R₁₁₅ is C₁-C₆alkyl;
R₁₁₆ is hydrogen, C₁-C₈alkyl, C₃-C₈alkenyl or C₃-C₈alkynyl, wherein the last 3 substituents may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₁₁₇ is hydrogen or C₁-C₈alkyl;
R₁₁₈ is hydrogen or C₁-C₈alkyl, or is C₁-C₈alkyl substituted by one or more —COOH, C₁-C₈-alkoxycarbonyl or —CN substituents; or
R₁₁₈ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₁₇ and R₁₁₈ together are C₂-C₅alkylene;
R₁₁₉ is hydrogen, C₁-C₄alkyl, C₁-C₄haloalkyl or C₃-C₆cycloalkyl; and
R₁₂₀ is hydrogen, C₁-C₄alkyl, C₁-C₄haloalkyl or C₃-C₆cycloalkyl;
R₁₂₁ is hydrogen, C₁-C₈alkyl, C₃-C₈alkenyl, C₃-C₈alkynyl, C₁-C₄haloalkyl or C₃-C₆haloalkenyl;
R₁₂₂ is hydrogen or C₁-C₈alkyl;
R₁₂₃ is hydrogen or C₁-C₈alkyl, or is C₁-C₈alkyl substituted by one or more —COOH, C₁-C₈-alkoxycarbonyl or —CN substituents; or
R₁₂₃ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₂₂ and R₁₂₃ together are C₂-C₅alkylene; and
R₁₂₄ is hydrogen or C₁-C₈alkyl,
or an agrochemically acceptable salt or any stereoisomer or tautomer of a compound of formula I.

2. A process for the preparation of a compound of formula I according to claim 1, which process comprises reacting a compound of formula II 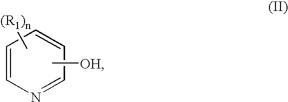
wherein R₁ and n are as defined in claim 1, in the presence of a base, with a compound of formula III 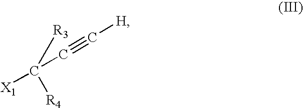
wherein R₃ and R₄ are as defined in claim 1 and X₁ is O-tosyl, O-mesyl, chlorine, bromine or iodine, to form a compound of formula IV 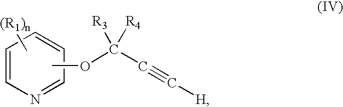
wherein R₁, R₃, R₄ and n are as defined in claim 1, and then coupling that compound with a compound of formula V 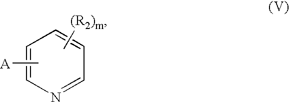
wherein R₂ and m are as defined in claim 1 and A is a leaving group such as, for example, halogen or trifluoromethanesulfonate, in the presence of a palladium catalyst.

3. A herbicidal and plant-growth-inhibiting composition, comprising a herbicidally effective amount of a compound of formula I according to claim 1 on an inert carrier.

4. A method of controlling undesired plant growth, which method comprises applying a compound of formula I according to claim 1, or a composition comprising such a compound, in a herbicidally effective amount to plants or to the locus thereof.

5. A method of inhibiting plant growth, which method comprises applying a compound of formula I according to claim 1, or a composition comprising such a compound, in a herbicidally effective amount to plants or to the locus thereof.

6. A compound according to claim 1, wherein R₄₁ is hydrogen, C₁-C₈alkyl, C₃-C₈alkenyl, C₃-C₈alkynyl, C₁-C₆alkoxy-C₁-C₆alkyl, C₁-C₈alkylcarbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or
R₄₁ is phenyl or phenyl-C₁-C₆alkyl, wherein the phenyl ring may in turn be substituted by one or more halogen, C₁-C₄alkyl, C₁-C₄haloalkyl, C₁-C₄alkoxy, —CN, —NO₂ or —S(O)₂C₁-C₈alkyl substituents, or
R₄₁ is C₁-C₈alkyl substituted by one or more —COOH, C₁-C₈alkoxycarbonyl, C₁-C₆alkylamino, di(C₁-C₆alkyl)amino or —CN substituents.

7. A compound according to claim 1, wherein each R₁ independently of any others is halogen, —CN, —NO₂, —C(R₁₀)═NOR₁₁, —OR₁₃, —S(O)₂R₁₆, C₁-C₈alkyl or C₂-C₈alkyl substituted by one or more halogen or —CN substituents; n is 0, 1, 2, 3 or 4; and R₁₀, R₁₁, R₁₃ and R₁₆ are as defined in claim 1.

8. A compound according to claim 7, wherein n is 1, 2 or 3.

9. A compound according to claim 1, wherein the group 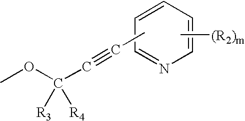
occupies the 2-position on the pyridine ring.

10. A compound according to claim 1, wherein each R₁ independently of any others is halogen, —CN, —NO₂, —C(R₁₀)═NOR₁₁, —OR₁₃, —SO₂R₁₆, C₁-C₈alkyl or C₂-C₈alkenyl; or C₁-C₈alkyl substituted by one or more halogen or —CN substituents; n is 1 or 2; R₁₀, R₁₁, R₁₃ and R₁₆ are as defined in claim 1; and the group 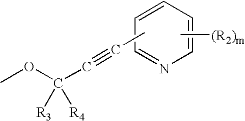
occupies the 2-position on the pyridine ring.

11. A compound according to claim 1, wherein each R₂ independently of any others is —CN, —SCN, —OCN, —N₃, —CONR₃₆R₃₇, —C(R₃₈)═NOR₃₉, —COR₄₀, —OR₄₁, —OSO₂R₄₅, —N([CO]ₚR₄₆)COR₄₇, —N(R₅₆)SO₂R₅₇, —N(SO₂R₅₈)SO₂R₅₉, —N═C(OR₆₀)R₆₁ or C₁-C₈alkyl; or C₁-C₈alkyl mono- or poly-substituted by halogen, —CN, —N₃, —SCN, —CONR₇₄R₇₅, —COR₇₆, —C(R₇₇)═NOR₇₈, —C(S)NR₇₉R₈₀, —OR₈₂, —SOR₈₄, —SO₂R₈₅ or by —N(R₈₉)COR₉₀; m is 0, 1, 2, 3 or 4; and R₃₆ to R₄₁ R₄₅ to R₄₇, R₅₆ to R₆₁, R₇₄ to R₈₀, R₈₂, R₈₄, R₈₅, R₈₉, R₉₀ and p are as defined in claim 1.

12. A compound according to claim 11, wherein m is 1 or 2.
